(19) United States
(12) Patent Application Publication
(10) Pub. No.: US 2006/0068441 A1
(43) Pub. Date: Mar. 30, 2006

(54) Alternative pol kappa nucleotide and amino acid sequence and methods for using

(76) Inventor: Juan Saus, Valencia (ES)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/274,658

(22) Filed: Nov. 15, 2005

Related U.S. Application Data
(63) Continuation of application No. 10/010,920, filed on Dec. 7, 2001.
(60) Provisional application No. 60/254,649, filed on Dec. 8, 2000.

(51) Int. Cl. C12Q 1/68; G01N 33/574; C07H 21/04; C12P 21/06; C07K 16/30; C07K 14/82; C40B 40/08

(52) U.S. Cl. 435/6; 435/7.23; 435/69.1; 435/320.1; 435/325; 530/350; 530/388.8; 536/23.5

(57) ABSTRACT

The present invention provides isolated nucleic acid sequences and expression vectors encoding the pol κC76, substantially purified pol κ76, and methods for detecting pol κ76.

### CROSS REFERENCE

This application claims priority to U.S. provisional application Ser. No. 60/254,649, filed Dec. 8, 2001.

### FIELD OF THE INVENTION

The present invention relates to the fields of gene regulation, autoimmunity, cancer, and apoptosis.

### BACKGROUND OF THE INVENTION

Goodpasture antigen binding protein (GPBP), is a non-conventional protein kinase that binds to and phosphorylates the human α3(IV)NC1 in vitro. [1,2,3] Its expression is associated with cells and tissue structures that are target of common autoimmune responses, including the alveolar and glomerular basement membranes [3]. GPBP Δ26 is an alternatively spliced GPBP variant, which is less active than GPBP, but more widely expressed [3]. A balanced expression of the two isoforms appears to be critical for homeostasis, whereas an augmented expression of GPBP relative to GPBPΔ26 has been associated with several autoimmune conditions, including Goodpasture disease and cutaneous lupus[3].

GPBP is expressed at very low levels in cancer cells and highly expressed in apoptotic blebs of differenced keratinocytes at the periphery of normal epidermis [3]. Keratinocytes from patients suffering skin autoimmune processes show an increased sensitivity to UV-induced apoptosis, and a premature apoptosis at the basal keratinocytes has been reported to occur in these patients [38-41]. GPBP is expressed in apoptotic bodies expanding from basal to peripheral strata in epidermis undergoing autoimmune attack [3]. Altered autoantigens, including phosphorylated versions thereof, have been reported to be produced and released from these apoptotic bodies [40]. All these data suggest that GPBP is part of an apoptotic-mediated strategy for desired cell removal that generates aberrant counterparts of critical cell components which operates illegitimately during autoimmune pathogenesis [3].

Pol κ is a member of the UmuC/DinB superfamily of DNA polymerases that can extend aberrant replication forks. Pol κ displays low fidelity, moderate processivity, and extends mispaired DNA by misaligning primer-template to generate −1 frameshift products [4-9]. Pol κ can bypass DNA lesions in both an error-prone [10,11] and an error-free [10] manner. These data indicate that pol κ is a DNA polymerase with a role in the cellular response to DNA-damage, and also in spontaneous mutagenesis, by facilitating base pairing at aberrant replication forks.

In the present study, we have determined that the structural genes encoding polK and GPBP are present in a head-to-head arrangement in the human genome at chromosome position 5q12-13, and that the genes share a common promoter from which the corresponding transcripts are expressed in a divergent mode. Our results demonstrate that TNF (α/β) induces divergent transcription directed by this promoter, suggesting that bi-directional promoters link the expression of proteins that are partners in biological programs which are orchestrated by TNF and are relevant in autoimmune pathogenesis. Furthermore we report the molecular cloning of pol κ76 an alternatively spliced variant preferentially expressed in skin and keratinocytes which like GPBP shows a relative augmented expression in cutaneous lupus, suggesting that pol κ76 and GPBP are partners in apoptotic programs which are relevant in autoimmune pathogenesis.

### SUMMARY OF THE INVENTION

The present invention provides an isolated nucleic acid encoding pol κ76 consisting of the nucleic acid sequence of SEQ ID NO:30, or the complement thereof. In another embodiment of this aspect, the present invention provides an isolated and purified pol κ76 protein consisting of the amino acid sequence of SEQ ID NO:31.

In a further embodiment of this aspect, the present invention provides a method for detecting an autoimmune condition in a patient, comprising providing a tissue or body fluid sample from the patient; providing a control tissue or body fluid sample in which no autoimmune condition is present; and detecting altered pol κ76 RNA or protein expression in the tissue or body fluid sample compared to the control sample, wherein an alteration in pol κ76 RNA or protein expression relative to the control indicates the presence of an autoimmune condition.

In a further embodiment, the present invention provides a method for treating a patient with an autoimmune disorder or cancer, comprising modifying the expression or activity of pol κ76 RNA or protein in the patient with the autoimmune disorder.

### BRIEF DESCRIPTION OF THE FIGURES

FIG. 1. Head-to-head arrangement of human POLK and COL4A3BP. The 955-bp between ON-GPBP-18m and ON-GPBP6c (GenBank accession no AF315603) (SEQ ID NO:2) are written in capital letters. In boldface the position and sequence of the two oligonucleotides, the restriction sites used to generate LpromPolK, LpromGPBP, or the construct from which the ribonucleotide probes are derived, and the DNA sequences which conform to the transcriptional elements identified by the TFSEARCH version 1.3. This DNA fragment contains the first exon of POLK (box), part of the first exon of COL4A3BP and the exon sequence of POLK contained in HeLa 4.1 (open boxes). The 5′ end and the transcriptional direction of HeLa 4.1. are indicated with arrows. The 1 40-bp present in SpromPolk and SpromGPBP is highlighted in gray.

FIG. 2. The POLK/COL4A3BP intergene region contains a bi-directional promoter. In A, NIH 3T3 cells were transfected with either pΦGH, Lprom (L bars), or Sprom (S bars) constructs, along with the β-galactosidase expressing vector. Results are expressed as the quotient (fold) of the reporter gene expression of the promoter constructs versus empty vector (pΦGH) after normalization with the corresponding β-galactosidase expression values. We represent the mean of two independent experiments done in duplicate, ±S.D. In B, NIH 3T3 cells were transfected as in A with SpromGPBP or SpromPolκ(wt), or with mutants thereof in which the TATA box (ΔTATA), the Sp1 site (ΔSp1), or both (ΔSpTA) were deleted. Transcriptional activity was estimated as in A and results are expressed as percent activity with respect to the wild type promoter, which was set at 100%, and are the mean ±S.D of three experiments done in duplicate.

FIG. 3. Alignment of each orientation of the 140-bp POLK/COL4A3BP promoter region with the corresponding regions of COL4A genes. In the Table we show the parameters of each individual alignment and those significant are shown in the map therein. Nucleotide numbering and map represent the DNA according to the GenBank accession numbers and the bend arrows mark the position and direction of the transcription start sites of the indicated gene.

FIG. 4. Alignment of each orientation of the 140-bp POLK/COL4A3BP promoter region with the corresponding regions of other bi-directional promoters. In the Table we show the parameters of each individual alignment and those significant less that of IDGH-TRAP which maps 3′ end of TRAP are shown in the map therein. Nucleotide numbering and map represent the DNA according to the GenBank accession numbers and the bend arrows mark the position and direction of the transcription start sites of the indicated gene.

FIG. 5. TNFα/β induce the 140-bp promoter of POLK/COL4A3BP and the homologous regions in other bidirectional promoters in transient gene expression assays. In A, NIH 3T3 cells were transfected with SpromPolk and SpromGPBP constructs along with β-galactosidase expressing vector and cells were induced with recombinant human counterparts of TNFα (10 ng/ml) or TNFβ (50 ng/ml). Results are expressed as the quotient (fold) of the reporter gene expression of the induced versus non-induced promoter constructs previous normalization with the corresponding β-galactosidase expression values. We represent the mean of four independent experiments done by duplicated ±S.D. In B, we represent the nucleotide sequence of the COL4A3/COL4A4 contained in AF218541 (SEQ ID NOS:8-13) as in the alignment map of FIG. 3 and we indicate the nucleotide which transcriptional activity was assayed as in A. For these purposes the indicated nucleotides from AF218541 in the indicated transcriptional orientation were individually transfected and further induced as in A. Results are expressed as reporter gene expression in c.p.m. (counts per minute) after normalization with β-galactosidase activity. We represent the mean of three independent experiments done by duplicated ±S.D. In C, the region of HSP10/HSP60 (SEQ ID NOS:2627) or LMP2/TAP1 (SEQ ID NOS:14-15) homologous with the COL4A3BP orientation of POLK/COL4A3BP promoter (FIG. 4) were individually cloned and assayed as in B.

FIG. 6. TNF induction of multiple bidirectional transcriptional units in human hTERT-RPE cells. Human hTERT-RPE cells, which are retinal pigment epithelial cells immortalized by over-expression of telomerase (Clontech) were induced by TNFβ, RNA was extracted and the transcriptional activity for the indicated genes estimated by specific mRNA quantification using the Relative Quantitation Method or “ΔΔCt” as described in Materials and Methods. The values represent fold induction of induced versus non-induced cells after normalization with GAPDH mRNA values and are the mean of three different samples done by duplicated ±S.D. The mRNA levels for GAPDH were not affected by cytokine induction.

FIG. 7. Evidences for increases in the relative expression of GPBP in response to TNF in vivo. B6 mice were injected with LPS and after three or six hours the kidneys were excised, total RNA prepared and the expression level of GPBP and GPBPΔ26 determined by Real Time PCR. Non-injected mice were used in control studies. Values represent the mean ±S.D. of two mice and four independent determinations.

FIG. 8. The relative increase of GPBP expression in response to TNF is a phenomenon with pathogenic consequences in a lupus prone mice model. In A, the kidney of female NZW, a male B6-Bcl-2-Tg(+) were paraffin-embedded and stained with GPBP-specific antibodies or mRNA prepared and the ratio of GPBP/GPBPΔ26 determined as in FIG. 7. The presence of glomerulonephritis (GN) in the kidneys was evaluated histologically according to glomerular celulariry and graded from absence (−) to discrete (+) moderate (++) or severe (+++). In B, the kidneys of (NZW×B6)F1Tg(+) mice treated with anti-CD4 (αCD4), treated with anti-CD4 and further maintained without treatment (αCD4/Ø), or treated with anti-CD4 and further treated with anti-TNF (αCD4/αTNF) were analyzed as in A. In A we present representative stainings and average values for GPBP/GPBPΔ26 whereas in B we present two examples for each case (N°1,2,3,4,10 and 14) in which one kidney was used for mRNA determinations and other for morphological studies. In C, the levels of anti-ssDNA autoantibodies in the sera of a number of six month old (NZW×B6Tg(+))F1 mice were determined by ELISA using an alkaline phosphatase-based conjugate. In the histogram each bar represent the values for each individual animal. Represented are non-trangenic F1 [F1Tg(−)], and transgenic F1 [F1Tg(+)] untreated (Ø) or treated with anti-CD4 for three month and then untreated [αCD4/Ø] or treated with anti-CD4 for three month and then treated with anti-TNF [αCD4/αTNF].

FIG. 9. Pol κ76 is a novel alternatively spliced form of pol κ preferentially expressed in keratinocytes which interacts with GIP a tumor suppressor gene product also interacting with GPBP In A, we schematized in a diagram the structural features of pol κ76 in comparison with pol κ. The predicted coiled-coil motifs (CC1 and CC2) previously unrecognized, and the features described in Ref. 5 for pol κ including nucleotidyl transferase domain (N), helix-haipin-helix (HhH1-2) and Zn cluster (Zn-c11 and Zn-c12) are indicated. The protein region of pol κ not present in pol κ76 is denoted by the convergent lines. In B, the mRNA levels for pol κ76 and for all of the pol κ molecular species known were estimated by Real Time PCR as described in Material and Methods in the indicated human cells and tissues. Values are expressed as the percentage of pol κ76 with respect total pol κ. With (Ø) we represent the non-specific amplification of pol κ standard plasmid using the pair of oligonucleotides employed for pol κ76 quantification. Values represent the mean ±S.D. of four determinations done on two different samples.

FIG. 10. The relative expression of pol κ76 and GPBP with respect to their alternative isoforms pol κ and GPBPΔ26 is augmented in cutaneous lupus. The expression of pol κ76, pol κ, GPBP and GPBPΔ26 was determined by Real Time PCR in reverse transcriptase mixtures of human foreskin (Control) or skin affected of cutaneous lupus (Patient 1-3). The indicated ratio values were normalized with respect to control ratio values that were set at 1. Values represent the mean ±S.D. of two determinations. In addition to clinical diagnosis all the patients samples had histological diagnosis confirmation and showed lineal deposits of immunocomplexes at the dermal-epidermal junction in direct immunofluorescence, which is characteristic of cutaneous lupus.

### DETAILED DESCRIPTION OF THE INVENTION

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the term “COL4A3BP” means the genomic sequence encoding GPBP, as well as controlling sequences for GPBP mRNA expression.

As used herein, the term “POLK” means the genomic sequence encoding pol κ, as well as controlling sequences for pol κ mRNA expression.

As used herein, the term “GPBP” refers to Goodpasture antigen binding protein, and includes both monomers and oligomers thereof, as disclosed in WO 00/50607.

As used herein, the term “GPBPΔ26” refers to the Goodpasture antigen binding protein alternatively spliced product deleted for 26 amino acid residues as disclosed in WO 00/50607, and includes both monomers and oligomers thereof.

As used herein pol κ means the primary protein product of the POLK.

As used herein, pol κ76 means the 76 kDa alternatively spliced isoform product of the POLK.

In one aspect, the present invention provides an isolated nucleic acid encoding a pol κ76 polypeptide consisting of an amino acid sequence of SEQ ID NO:31. In a preferred embodiment, the isolated nucleic acid consists of the sequence of SEQ ID NO:30, the complement thereof, or the RNA expression product thereof.

An used herein, an “isolated nucleic acid sequence” refers to a nucleic acid sequence that is free of gene sequences which naturally flank the nucleic acid in the genomic DNA of the organism from which the nucleic acid is derived (i.e., genetic sequences that are located adjacent to the gene for the isolated nucleic molecule in the genomic DNA of the organism from which the nucleic acid is derived). An “isolated” pol κ76 nucleic acid sequence according to the present invention may, however, be linked to other nucleotide sequences that do not normally flank the recited sequence, such as a heterologous promoter sequence, or other vector sequences. It is not necessary for the isolated nucleic acid sequence to be free of other cellular material to be considered “isolated”, as a nucleic acid sequence according to the invention may be part of an expression vector that is used to transfect host cells (see below).

In another embodiment, the present invention provides an expression vector comprising an isolated nucleic acid encoding pol κ76 operatively linked to a promoter, wherein the isolated nucleic acid consists of the sequence of SEQ ID NO:30. In a preferred embodiment, the promoter is heterologous (i.e.: is not the naturally occurring POLK promoter).

A promoter and a pol κ76 nucleic acid sequence are “operatively linked” when the promoter is capable of driving expression of the pol κ76 DNA into RNA.

As used herein, the term “vector” refers to a nucleic acid molecule capable of transporting another nucleic acid to which it has been linked. One type of vector is a “plasmid”, which refers to a circular double stranded DNA into which additional DNA segments may be cloned. Another type of vector is a viral vector, wherein additional DNA segments may be cloned into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors), are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In the present invention, the expression of any genes is directed by the promoter sequences of the invention, by operatively linking the promoter sequences of the invention to the gene to be expressed. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The vector may also contain additional sequences, such as a polylinker for subcloning of additional nucleic acid sequences, a polyadenylation signal to effect proper polyadenylation of the transcript. The nature of the polyadenylation signal is not believed to be crucial to the successful practice of the invention, and any such sequence may be employed, including but not limited to the SV40 and bovine growth hormone poly-A sites. Also contemplated as an element of the vector is a termination sequence, which can serve to enhance message levels and to minimize read through from the construct into other sequences. Finally, expression vectors typically have selectable markers, often in the form of antibiotic resistance genes, that permit selection of cells that carry these vectors.

In a further embodiment, the present invention provides recombinant host cells transfected with the expression vectors disclosed herein. As used herein, the term “host cell” is intended to refer to a cell into which a nucleic acid of the invention, such as a recombinant expression vector of the invention, has been introduced. Such cells may be prokaryotic, which can be used, for example, to rapidly produce a large amount of the expression vectors of the invention, or may be eukaryotic, for functional studies.

The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The host cells can be transiently or stably transfected with one or more of the expression vectors of the invention. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.).

In another embodiment, the present invention provides an isolated and purified pol κ76 polypeptide consisting of the amino acid sequence of SEQ ID NO:31.

As used herein, an “isolated polypeptide” refers to a polypeptide that is substantially free of other proteins, cellular material and culture medium when isolated from cells or produced by recombinant DNA techniques, or chemical precursors or other chemicals when chemically synthesized. Thus, the protein can either be purified from natural sources, or recombinant protein can be purified from the transfected host cells disclosed above. In a preferred embodiment, the proteins are produced by the transfected cells disclosed above, and purified using standard techniques. (See for example, Molecular Cloning: A Laboratory Manual (Sambrook, et al., 1989, Cold Spring Harbor Laboratory Press.)) The protein can thus be purified from prokaryotic or eukaryotic sources. In various further preferred embodiments, the protein is purified from bacterial, yeast, or mammalian cells.

The protein may comprise additional sequences useful for promoting purification of the protein, such as epitope tags and transport signals. Examples of such epitope tags include, but are not limited to FLAG (Sigma Chemical, St. Louis, Mo.), myc (9E10) (Invitrogen, Carlsbad, Calif.), 6-His (Invitrogen; Novagen, Madison, Wis.), and HA (Boehringer Manheim Biochemicals). Examples of such transport signals include, but are not limited to, export signals, secretory signals, nuclear localization signals, and plasma membrane localization signals.

In a further aspect, the invention provides methods for detecting the presence of the pol κ76 in a protein sample, comprising providing a protein sample to be screened, contacting the protein sample to be screened with an antibody against pol κ76, or another compound that specifically interacts with pol κ76, and detecting the formation of antibody-antigen or pol κ76-compound complexes. Where the detection is done via antibody detection, the antibody can be either polyclonal or monoclonal, although monoclonal antibodies are preferred. As used herein, the term “protein sample” refers to any sample that may contain pol κ76, including but not limited to tissues and portions thereof, tissue sections, intact cells, cell extracts, purified or partially purified protein samples, bodily fluids, nucleic acid expression libraries. Accordingly, this aspect of the present invention may be used to test for the presence of pol κ76 antigen in these various protein samples by standard techniques including, but not limited to, immunolocalization, immunofluorescence analysis, Western blot analysis, ELISAs, aid nucleic acid expression library screening, (See for example, Sambrook et al, 1989.) In one embodiment, the techniques may determine only the presence or absence of pol κ76. Alternatively, the techniques may be quantitative, and provide information about the relative amount of pol κ76 in the sample. For quantitative purposes, ELISAs are preferred.

Detection of immunocomplex formation between pol κ76 and antibodies or fragments thereof, directed against pol κ76, can be accomplished by standard detection techniques. For example, detection of immunocomplexes can be accomplished by using labeled antibodies or secondary antibodies. Such methods, including the choice of label are known to those ordinarily skilled in the art. (Harlow and Lane, Supra). Alternatively, the polyclonal or monoclonal antibodies can be coupled to a detectable substance. The term “coupled” is used to mean that the detectable substance is physically linked to the antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase. Examples of suitable prosthetic-group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerytirin. An example of a luminescent material includes luminol. Examples of suitable radioactive material include ¹²⁵I, ¹³¹I, ³⁵S or ³H.

Such methods of detection are useful for a variety of purposes, including but not limited to detecting an autoimmune condition, identifying cells targeted for or undergoing apoptosis, immunolocalization of pol κ76 in a tissue sample, Western blot analysis, and screening of expression libraries to find related proteins.

Antibodies can be made by well-known methods, such as described in Harlow and Lane, Antibodies; A Laboratory Manual, Cold Spring Harbor Laboratory, Cold Spring Harbor, N.Y., (1988). In one example, preimmune serum is collected prior to the first immunization. A peptide portion of the amino acid sequence of pol κ76 that is not co-linear in pol κ, due to the alternative splicing of the pre-mRNA, together with an appropriate adjuvant, is injected into an animal in an amount and at intervals sufficient to elicit an immune response. Animals are bled at regular intervals, preferably weekly, to determine antibody titer. The animals may or may not receive booster injections following the initial immunization. At about 7 days after each booster immunization, or about weekly after a single immunization, the animals are bled, the serum collected, and aliquots are stored at about −20° C. Polyclonal antibodies against pol κ76 can then be purified directly by passing serum collected from the animal through a column to which non-antigen-related proteins prepared from the same expression system without pol κ76 bound.

Monoclonal antibodies can be produced by obtaining spleen cells from the animal. (See Kohler and Milstein, Nature 256, 495-497 (1975)). In one example, monoclonal antibodies (mAb) of interest are prepared by immunizing inbred mice with peptide portion of the amino acid sequence of pol κ76 that is not co-linear in pol κ, due to the alternative splicing of the pre-mRNA. The mice are immunized by the IP or SC route in an amount and at intervals sufficient to elicit an immune response. The mice receive an initial immunization on day 0 and are rested for about 3 to about 30 weeks. Immunized mice are given one or more booster immunizations of by the intravenous (IV) route. Lymphocytes, from antibody positive mice are obtained by removing spleens from immunized mice by standard procedures known in the art. Hybridoma cells are produced by mixing the splenic lymphocytes with an appropriate fusion partner under conditions which will allow the formation of stable hybridomas. The antibody producing cells and fusion partner cells are fused in polyethylene glycol at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells and are screened for antibody production by an immunoassay such as solid phase immunoradioassay. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

To generate such an antibody response, the peptide portion of the amino acid sequence of pol κ76 that is not co-linear in pol κ, is typically formulated with a pharmaceutically acceptable carrier for parenteral administration. Such acceptable adjuvants include, but are not limited to, Freund's complete, Freund's incomplete, alum-precipitate, water in oil emulsion containing Corynebacterium parvum and tRNA. The formulation of such compositions, including the concentration of the polypeptide and the selection of the vehicle and other components, is within the skill of the art.

The term antibody as used herein is intended to include antibody fragments thereof which are selectively reactive with pol κ76. Antibodies can be fragmented using conventional techniques, and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

In yet another aspect, the invention provides methods for detecting the presence in a sample of nucleic acid sequences encoding pol κ76 comprising providing a nucleic acid sample to be screened, contacting the sample with a nucleic acid probe derived from the isolated nucleic acid sequences of the invention, or fragments thereof, and detecting complex formation.

As used herein, the term “sample” refers to any sample that may contain pol κ76 -encoding nucleic acid, including but not limited to tissues and portions thereof, tissue sections, intact cells, cell extracts, purified or partially purified nucleic acid samples, DNA libraries, and bodily fluids. Accordingly, this aspect of the present invention may be used to test for the presence of pol κ76 mRNA or DNA in these various samples by standard techniques including, but not limited to, in situ hybridization, Northern blotting, Southern blotting, DNA library screening, polymerase chain reaction (PCR) or reverse transcription-PCR (RT-PCR). (See for example, Sambrook et al, 1989.) In one embodiment, the techniques may determine only the presence or absence of the nucleic acid of interest. Alternatively, the techniques may be quantitative, and provide information about the relative amount of the nucleic acid of interest in the sample. For quantitative purposes, quantitative PCR and RT-PCR are preferred. Thus, in one example, RNA is isolated from a sample, and contacted with an oligonucleotide derived from the pol κ76 nucleic acid sequence, together with reverse transcriptase under suitable buffer and temperature conditions to produce cDNAs from the pol κ76 RNA. The cDNA is then subjected to PCR using primer pairs derived from the nucleic acid sequence of interest. In a preferred embodiment, the primers are designed to detect the presence of the RNA expression product of SEQ ID NO:30, and the amount of pol κ76 gene expression in the sample is compared to the level in a control sample.

For detecting pol κ76 nucleic acid sequences, standard labeling techniques can be used to label the probe, the nucleic acid of interest, or the complex between the probe and the nucleic acid of interest, including, but not limited to radio-, enzyme-, chemiluminescent-, or avidin or biotin-labeling techniques, all of which are well known in the art. (See, for example, Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.); PCR Protocols: A Guide to Methods and Applications (Innis, et al. 1990. Academic Press, San Diego, Calif.)).

Such methods of nucleic acid detection are useful for a variety of purposes, including but not limited to diagnosing an autoimmune condition, identifying cells targeted for or undergoing apoptosis, in situ hybridization, Northern and Southern blot analysis, and DNA library screening.

As demonstrated in the following examples, pol κ76 shows preferential expression in skin and keratinocytes that are commonly targeted in naturally-occurring automimmune responses, and is expressed at an elevated level in systemic lupus erythematosus (SLE) patients. Furthermore, pol κ76 is shown herein to be associated through another protein with GPBP, which is known to be associated with autoimmune conditions.

Thus, in a preferred embodiment, detection of pol κ76 expression is used to detect an autoimmune condition. A sample that is being tested is compared to a control sample for the expression of pol κ76 RNA, wherein an increased level of pol κ76 RNA expression indicates the presence of an autoimmune condition. In this embodiment, it is preferable to use quantitative RT-PCR and to employ oligonucleotides that generate a product from pol κ76 RNA but not polK RNA, or that produce distinguishable products from pol κ76 RNA and pol κ RNA.

Furthermore, pol κ76/pol K are likely to be involved in cell signaling pathways that induce apoptosis, which may be up-regulated during autoimmune pathogenesis and down-regulated during cell transformation to prevent autoimmune attack to transformed cells during tumor growth. Thus, the detection methods disclosed herein can be used to detect cells that are targeted for, or are undergoing apoptosis.

In another aspect, the present invention provides a method for treating an autoimmune disorder or cancer comprising modification of the expression or activity of pol κ76 RNA or pol κ76 polypeptide, such as by increasing or decreasing their expression or activity. Modifying the expression of or activity of pol κ76 RNA or pol κ76 polypeptide can be accomplished by using specific inducers or inhibitors of pol κ76 expression or activity, pol κ76 antibodies, antisense therapy, or other techniques known in the art. As used herein, “modification of expression or activity” refers to modifying expression or activity of either the RNA or protein product.

The present invention may be better understood with reference to the accompanying examples that are intended for purposes of illustration only and should not be construed to limit the scope of the invention, as defined by the claims appended hereto.

### EXAMPLES

Materials and Methods.

Synthetic oligonucleotides. The following oligonucleotides and other used for DNA sequencing were synthesized by Genosys, Life Technology Inc., Roche or Pharmacia:

| | | |
|---|---|---|
| ON-GPBP-6c, | CTCGCTCGCCCAGGGAAGGAAAAGGGAAAAGAAGGGA-3′; | (SEQ ID NO:37) |
| ON-GPBP-14c, | 5′-CTGCCTGGCCCACTATTTAGC-3′; | (SEQ ID NO:38) |
| ON-GPBP-18m, | 5′-GGCATGGTTAACGTGGTTCTC-3′; | (SEQ ID NO:39) |
| ON-XbaG/Bpro1m, | 5′-GACTCTAGAGGGTTCGGGAGGAGGATCCCG-3′; | (SEQ ID NO:40) |
| ON-XbaG/Bpro1c, | 5′-GACTCTAGACTGGCCCACTATTTACCCTCC-3′; | (SEQ ID NO:41) |
| ON-SP1Del, | 5′-CGCCGGGAGGGGGACGTAGTGGGGGAGAAT-3′; | (SEQ ID NO:42) |
| ON-TATADel, | 5′-CAGGGGAGGGGAGGGGTGGGCCAGTCTAGA-3′; | (SEQ ID NO:43) |
| ON-DIN2c, | 5′-GGATTATTGCACTTGCCTTCAC-3′; | (SEQ ID NO:44) |
| ON-DIN5′m, | 5′-AAAGGATCCATGGATAGCACAAAGGAG-3′; | (SEQ ID NO:45) |
| ON-DIN-THc, | 5′-AAAAAAGTCGACTTAGTTAAAAAATATATCAAGGGT-3′; | (SEQ ID NO:46) |
| ON-D1NB1-R2, | 5′-TGGTATTGCTCAAATTTCGGC-3′; | (SEQ ID NO:47) |
| ON-GPBP-39c, | 5′-TGAGAGAGCTTTCCGCTG-3′; | (SEQ ID NO:48) |
| ON-LMPTAP1m, | 5′-ATGTCTAGATGTGTAGGGCAGATCTGCCC-3′; | (SEQ ID NO:49) |
| ON-LMPTAP1c, | 5′-ATGTCTAGACTGGTGCCCAATTTTCTCCA-3′; | (SEQ ID NO:50) |
| ON-HSP1m, | 5′-ATGTCTAGATAAGCCGGCCGGAGAGGGCT-3′; | (SEQ ID NO:51) |
| ON-HSP1c, | 5′-ATGTCTAGACGCGGCACCGCGTGTGCAGG-3′; | (SEQ ID NO:52) |
| ON-SA3A4m, | 5′-GACTCTAGAGGGTTAAGGAGGTGATGCTCCC-3′; | (SEQ ID NO:53) |
| ON-SA3A4c, | 5′-GACTCTAGATGGCCACTCCCTCCACCCTGCGC-3′; | (SEQ ID NO:54) |
| ON-INGA3A4m, | 5′-GACTCTAGACACCCAGGCTTTTTGGTTGTGGC-3′; | (SEQ ID NO:55) |
| ON-INGA3A4c, | 5′-GACTCTAGAAAGCGGGGCCTCCCGCAGACGC-3′; | (SEQ ID NO:56) |
| ON-S2A3A4m, | 5′-ATGTCTAGATAGGCACTGGACAAGCCCCC-3′; | (SEQ ID NO:57) |
| ON-S2A3A4c, | 5′-ATGTCTAGAGGGCTAGTGGCGAGGCTGAG-3′; | (SEQ ID NO:58) |
| ON-IDH-F1, | 5′-CACAGAGGGCGAGTACAGCA-3′; | (SEQ ID NO:59) |
| ON-IDH-R1, | 5′-TGATCTTCAGGCTCTCCACCA-3′; | (SEQ ID NO:60) |
| ON-TRAPD-F1, | 5′-GGGTCCAGAACATGGCTCTC-3′; | (SEQ ID NO:61) |
| ON-TRAPD-R1, | 5′-ACATCCTGGCCTCGAGTGAC-3′; | (SEQ ID NO:62) |
| ON-LMP2-F2, | 5′-GCAGCATATAAGCCAGGCATG-3′; | (SEQ ID NO:63) |
| ON-LMP2-R2, | 5′-TGGCCAGAGCAATAGCGTCT-3′; | (SEQ ID NO:64) |
| ON-TAP1-F2, | 5′-GCCGCCTCACTGACTGGAT-3′; | (SEQ ID NO:65) |
| ON-TAP1-R2, | 5′-TCGAGTGAAGGTATCGGCTGA-3′; | (SEQ ID NO:66) |
| ON-DHFR-F1, | 5′-CCTGTGGAGGAGGAGGTGG-3′; | (SEQ ID NO:67) |
| ON-DHFR-R1, | 5′-CCGATTCTTCCAGTCTACGGG-3′; | (SEQ ID NO:68) |
| ON-MSH3-F1, | 5′-TGGGTAAAGGTTGGAAGCACA-3′; | (SEQ ID NO:69) |
| ON-MSH3-R1, | 5′-AAAAGGAGAGTGAAAGCGGCT-3′; | (SEQ ID NO:70) |
| ON-HO3-F2, | 5′-GAGCTGTTGTCCCTCCGCT-3′; | (SEQ ID NO:71) |
| ON-HO3-R2, | 5′-GGCCAGATAACGAGCAAAGG-3′; | (SEQ ID NO:72) |
| ON-HARS-F2, | 5′-AGGTGGCGAAACTCCTGAAAC-3′; | (SEQ ID NO:73) |
| ON-HARS-R2, | 5′-TGCTTTCATCAGGACCCAGC-3′; | (SEQ ID NO:74) |
| ON-Hsp10-F1, | 5′-GGAGGGAGTAATGGCAGGACA-3′; | (SEQ ID NO:75) |
| ON-Hsp10-R1, | 5′-AGCAGCACTCCTTTCAACCAA-3′; | (SEQ ID NO:76) |
| ON-Hsp60-F1, | 5′-GCCTTTGGTCATAATCGCTGA-3′; | (SEQ ID NO:77) |
| ON-Hsp60-R1, | 5′-TGCCACAACCTGAAGACCAAC-3′; | (SEQ ID NO:78) |
| ON-COL4A1-F1, | 5′-GCTCTACGTGCAAGGCAATGA-3′; | (SEQ ID NO:79) |
| ON-COL4A1-R1, | 5′-ATTGTGCTGAACTTGCGCAG-3′; | (SEQ ID NO:80) |
| ON-COL4A2-F1, | 5′-GAAAAGGGTGACGTAGGGCA-3′; | (SEQ ID NO:81) |
| ON-COL4A2-R1, | 5′-GGTGTCTGATGGAATCCCGTT-3′; | (SEQ ID NO:82) |
| ON-GP-F1, | 5′-GGAGACAGTGGATCACCTGCA-3′; | (SEQ ID NO:83) |
| ON-GP-R1, | 5′-TGCTGTGGTTTGACTGTGTCG-3′; | (SEQ ID NO:84) |
| ON-COL4A4-F1, | 5′-CTTGCCTTCCCGTATTTAGCA-3′; | (SEQ ID NO:85) |
| ON-COL4A4-R1, | 5′-GGATCTGTCGTTTCTCTGGGC-3′; | (SEQ ID NO:86) |
| ON-COL4A5-F1, | 5′-CATCGAATGTCATGGGAGGG-3′; | (SEQ ID NO:87) |
| ON-COL4A5-R1, | 5′-AGTTGCCAGCCAAAAGCTGTA-3′; | (SEQ ID NO:88) |
| ON-COL4A6-F1, | 5′-TTTGGGCTAGACTACCGGACA-3′; | (SEQ ID NO:89) |
| ON-COL4A6-R1, | 5′-TCTCTATGGACCCGAGGGCT-3′; | (SEQ ID NO:90) |
| ON-GPBP-F1, | 5′-CTGAATCCAGCTTGCGTCG-3′; | (SEQ ID NO:91) |
| ON-GPBP-R1, | 5′-GCAGAGTAGCCACTTGCTCC-3′; | (SEQ ID NO:92) |
| ON-DinB1-F3, | 5′-GCCCCCCAACTTTGACAAAT-3′; | (SEQ ID NO:93) |
| ON-DinB1-R3, | 5′-GCTTCATCAAGACTCATGGCC-3′; | (SEQ ID NO:94) |
| ON-hGAPDH-F1, | 5′-GAAGGTGAAGGTCGGAGTC-3′; | (SEQ ID NO:95) |
| ON-hGAPDH-R1, | 5′-GAAGATGGTGATGGGATTTC-3′; | (SEQ ID NO:96) |
| ON-GPBP-26-1F, | 5′-GCTGTTGAAGCTGCTCTTGACA-3′; | (SEQ ID NO:97) |
| ON-mGPBP-26-1R, | 5′-CCATTTCTTCAACCTTTTGTACAA-3′; | (SEQ ID NO:98) |
| ON-GPBPe26-1R, | 5′-CTTGGGAGCTGAATCTGTGAA-3′; | (SEQ ID NO:99) |
| ON-huDINB-76-F1, | 5′-CCAGTGCAGGTGTTCGGATA-3′; | (SEQ ID NO:100) |
| ON-huDINB-76-R1, | 5′-TTTCCAGCCTGTAAAAAGCCA-3′. | (SEQ ID NO:101) |
| ON-hGPBP-26-1R, | 5′-CCATCTCTTCAACCTTTTGGACA-3′ | (SEQ ID NO:102) |

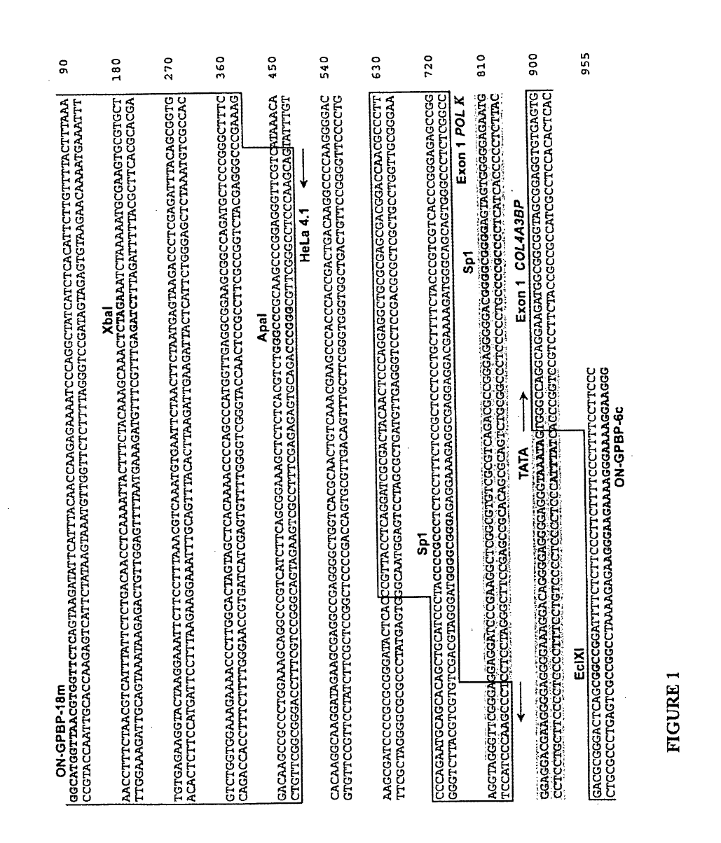

Isolation of the 5′ genomic region of COL4A3BP. The 5′-end region of COL4A3BP was isolated by PCR using ON-GPBP-6c, Adapter primer 2 (AP2)(Clontech) and DNA from human genomic libraries (PromoterFinder DNA Walking Kit (Clontech)). We obtained a single DNA fragment in four of the five of the libraries screened (1.6, 1.3, 0.8, and 0.4 kb, respectively). By sequencing the 0.4-kb DNA fragment we characterized the COL4A3BP region immediately upstream of the cDNA clone (n4′) (SEQ ID 1) previously reported (Disclosed in WO 00/50607; GenBank accession no AF136450) [2]. Based on the sequence of the 0.4 kb fragment, we designed and synthesized ON-GPBP-14c, and used it in combination with AP2 to perform PCR on the 1.6 kb genomic library fragment. From this PCR, we obtained a PCR DNA fragment of ˜1.5 kb containing the 5′ genomic region of COL4A3BP without any exon sequences present in n4′. This DNA fragment was then used to screen a HeLa-derived cDNA library, from which we isolated HeLa 4.1, a clone containing 1.3 kb of cDNA (SEQ ID NO:2 (GenBank accession no AF315601). Finally, we used ON-GPBP-18m (an oligonucleotide derived from HeLa 4.1) and ON-GPBP-6c (an oligonucleotide derived from n4′) to conduct PCR on human genomic DNA, from which we generated a 955-bp PCR product (SEQ ID NO:3)(GenBank accession no AF315603) that contained HeLa 4.1 sequence, the 5′ region of the first exon of COL4A3BP, and the intervening DNA region (FIG. 1).

Plasmid construction. A 772-bp DNA fragment was generated by digesting the 955-bp PCR product (SEQ. ID NO:3) with XbaI and Ec1XI, the ends were filled-in, and the orientation expressing COL4A3BP (SEQ ID NO:4) or POLK (SEQ ID NO:5) cloned into the HincII site of pΦGH (Nichols Institute) immediately upstream of human growth hormone reporter gene to generate LpromGPBP and LpromPolK. Alternatively, ON-XbaG/Bpro1m and ON-XbaG/Bprolc were used to obtain a 140-bp PCR product which contained the intergene region, the major transcription start sites for each gene and a few nucleotides of the corresponding exon 1 from either COL4A3BP or POLK (shaded sequence in FIG. 1). Upon digestion with XbaI, each of the two orientations (SEQ ID NO: 6; SEQ ID NO: 7) was cloned in the corresponding restriction site of the polylinker region of pΦGH to generate SpromGPBP and SpromPolic, respectively. Subsequently, SpromGPBP was used to obtain constructs in which Sp1, TATA, or both sites were selectively deleted. This was accomplished using ON-SP1Del, ON-TATADel or both and a site-directed mutagenesis approach. To obtain the corresponding promoter mutants for POLK, we cloned the reverse orientation of the SpromGPBP mutants by XbaI digestion and re-ligation.

To generate pΦGH-based constructs containing 140-bp homologous regions of COL4A3/COL4A4, LMP2/TAP1 and HSP10/HSP60, human DNA was prepared from blood cells using a DNA purification kit (Epicenter), and the regions of interest amplified by PCR using the following pair of synthetic oligonucleotides ON-S2A3A4m/ON-S2A3A4c, ON-SA3A4m/ON-SA3A4c, ON-INGA3A4m/ON-INGA3A4c to obtain the DNA regions corresponding to 182-318 (SEQ ID NO: 8; SEQ ID NO:9), 849-990 (SEQ ID NO: 10; SEQ ID NO:11), 675-1045 nucleotides (SEQ ID NO: 12; SEQ ID NO:13) of AF218541; ON-LMPTAP1m/ON-LMPTAP1c to obtain the DNA fragment containing the 24579-24718 nucleotides (SEQ ID NO: 14; SEQ ID NO:15) of X66401; and ON-HSP1m/ONHSP1c to obtain the 3451-3590 nucleotides (SEQ ID NO: 26; SEQ ID NO:27) of AJ250915. The DNA fragments were individually digested with XbaI and cloned in the corresponding site of the polylinker region of pΦDGH in each of the two orientations.

To generate pGBT9 and pGAD424 plasmids for pol κ and pol κ76 the corresponding cDNA fragments obtained by RT-PCR (see below) were digested with BamHI and SalI and cloned in the corresponding sites of a FLAG modified version of the corresponding expression vectors (Clontech) engineered essentially as previously described [2] but containing a BamHI site immediately downstream of the FLAG peptide sequence.

All the plasmid-based constructs were characterized by nucleotide sequencing.

Plasmid expressing human glyceraldehyde 3-phosphate dehydrogenase (GAPDH) was provided by Erwin Knecht.

Ribonuclease protection assays. By digesting LpromGPBP with ApaI and EclXI we obtained a DNA fragment of 503-bp containing the two 5′ end regions of POLK and COL4A3BP genes and the intergene region. The DNA fragment was blunt-end with T4 DNA polymerase and cloned into the HincII site of Bluescribe M13+ (Stratagene). Ribonucleotide probes from T3 and T7 promoters representing the antisense of the GPBP or pol κ mRNAS respectively were obtained using MAXIscript™ T7/T3 in vitro transcription kit (Ambion). Individual ribonucleotide probes were subject to ribonuclease protection assays using RPAIII™ (Ambion) and total RNA from human cultured hTERT-RPE1 (Clontech) or 293 cells (ATCC # CRL-1573). The digestion mixtures were analyzed by gel electrophoresis (8M urea 8% acrylamide gel) and autoradiography.

RNA purification. Total RNA was prepared from human tissues or cultured cells using TRI-REAGENT (Sigma) and following the manufacturer's recommendations.

Reverse transcription (RT) and polymerase chain reactions studies(PCR).

To obtain a continuous cDNA fragment containing HeLa 4.1 and pol κ coding sequences (GenBank accession no AF318313 (SEQ ID NO: 32) we carried out a PCR on human striated muscle cDNA library (MATCHMAKER™ from Clontech) with ON-GPBP-39c and ON-DINB1-R2 primers using the Expand™ Long Template PCR System (Roche). To obtain the cDNA for pol κ or pol κ76, 5 μg of total RNA extracted from human foreskin was reverse-transcribed with ON-DIN2c using the Ready-To-Go system (Pharmacia). An aliquot (0.5 μl) of the resulting cDNA-RNA hybrid was similarly subjected to PCR using ON-DIN5'm and ON-DIN-THc.

Real Time PCR studies were performed using a SDS 7700 Applied Biosystems apparatus and aliquots of either human cDNA libraries for striated muscle, HeLa cells, keratinocytes, pancreas, brain and kidney (MATCHMAKER from Clontech) or random hexamer reverse-transcriptase reactions performed as above using total RNA extracted from human hTERT-RPE1 cells, foreskin, lung, spleen, adrenal gland and kidney or from mouse kidney.

The mRNA determinations in hTERT-RPE were done on 5 μl of a 1:10 (for the different genes of interest) or 1:1000 (for GAPDH) dilution of a single reverse transcriptase reaction using the Relative Quantitation Method analysis (ΔΔCt) following manufacturer's recommendations. GAPDH was used as endogenous control to normalize quantification. The pair of oligonucleotides were, ON-IDH-F1 and ON-IDH-R1 for IDHG; ON-TRAPD-F1 and ON-TRAPD-R1 for TRAPD; ON-LMP2-F2 and ON-LMP2-R2 for LMP2; ON-TAP1-F2 and ON-TAP1-R2 for TAPI; ON-DHFR-F1 and ON-DHFR-R1 for DHFR; ON-MSH3-F1 and ON-MSH3-R1 for MRP1; ON-HO3-F2 and ON-HO3-R2 for HO3; ON-HARS-F2 and ON-HARS-R2 for HRS; ON-Hsp10-F1 and ON-Hsp10-R1 for HSP10; ON-Hsp60-F1 and ON-Hsp60-R1 for HSP60; ON-COL4A1-F1 and ON-COL4A1-R1 for COL4A1; ON-COL4A2-F1 and ON-COL4A2-R1 for COL4A2; ON-GP-F1 and ON-GP-R1 for COL4A3; ON-COL4A4-F1 and ON-COL4A4-R1 for COL4A4; ON-COL4A5-F1 and ON-COL4A5-R1 for COL4A5; ON-COL4A6-F1 and ON-COL4A6-R1 for COL4A6; ON-GPBP-F1 and ON-GPBP-R1 for COL4A3BP; ON-DinB1-F3 and ON-DinB1-R3 for POLK; ON-hGAPDH-F1 and ON-hGAPDH-R1 for GAPDH.

To determine mRNA levels for human pol κ or pol κ76 PCR reactions were performed using ON-DINB1-F3 and ON-DINB1-R3 or ON-huDINB-76-F1 and ON-huDINB-76-R1 respectively, and either 6 and 60 ng of the different cDNA libraries, or 5 μl of a 1:10 dilution of the individual reverse transcriptase reactions. Standard curves for each PCR were done using the same oligonucleotides and different amounts of individual plasmids containing the corresponding cDNAs.

To determine GPBP and GPBPΔ26 mRNA levels in mouse kidney PCR reactions were done using ON-GPBP-26-1F and ON-GPBPe26-1R or ON-mGPBP-26-1R, respectively and 5 μl of a 1:10 and 1:100 dilution of the individual reverse transcriptase reactions.

To determine GPBP and GPBPΔ26 mRNA levels in human skin samples PCR reactions were done using ON-GPBP-26-1F and ON-GPBPe26-1R or ON-hGPBP-26-1R, respectively and 5 μl of a 1:10 dilution of the individual reverse transcriptase reactions.

Northern analysis. Pre-made Northern blots (Clontech) were probed with ³²P-labeled cDNAs representing GPBP (n4′) or pol κ (see above) according to manufacturer's instructions.

Cell culture and transient gene expression assays. Cells were grown in DMEM (NIH 3T3 and 293) or DMEM F-12 HAM (hTERT-RPE1) with 100 units/ml of penicillin and 100 μg/ml streptomycin, and supplemented with 10% calf serun (NIH 3T3 cells) or fetal calf serum (hTERT-RPE1 and 293). For transient gene expression assays, NIH 3T3 cells (1.4×10⁵) were seeded in 9.5 cm² plates, cultured for 14-16 hours, and then transfected for 16-18 hours with 2.5 μg of each individual pΦGH-derived plasmid and 2.5 μg of β-galactosidase expression vector (Promega) using the calcium phosphate precipitation method of the Profection Mammalian Transfection System (Promega). After transfection, the cells were rinsed with phosphate-buffered saline, fresh medium was added, and the levels of human growth hormone in the media were determined after 48 hours using a solid phase radioimmunoassay system (Nichols Institute). β-galactosidase activity determination was performed following manufacturer's recommendations. For some purposes, after transfection the cells were cultured in low serum (0.5%) media for 24 hours, media was discarded, and fresh low serum media containing TNFα (10 ng/ml) or TNFβ(50 ng/ml) was added, and levels of human growth hormone similarly determined.

For other purposes hTERT-RPE1 cells were grown up to 60-70% confluence, media removed and fresh serum-free media added and culture continued. After 24 hours the media was removed, fresh serum-free media containing TNFβ (50 ng/ml) added, and, after one hour, the media was discarded and cells were used for RNA preparation.

Isolation of genomic DNA encoding GPBP. We have used human GPBP cDNA fragments obtained from specific PCR amplification of n4′ to screen a human genomic library, λfix-w138 (Stratagene). Two independent and overlapping genomic clones λfixGPBP1 and λfixGPBP3, of ˜14 kb and ˜13 kb respectively, were characterized by restriction mapping and partial nucleotide sequencing. The nucleotide sequence of ˜12 Kb of the λfixGPBP1 has been recently reported (GenBank accession no AF232935) [3].

Chromosome localization of COL4A3BP, the structural gene for GPBP. To map COL4A3BP, a fluorescence in situ hybridization (FISH) analysis was performed essentially as described in Ref. 13 on metaphase chromosomes obtained from control peripheral blood using λfixGPBP1 and λfixGPBP3, labeled by standard nick-translation with digoxigenin-11-dUTP and biotin-16-dUTP respectively. The hybridized material was detected using either sheep anti-digoxigenin-FITC (fluoresceine isothiocyanate (Roche) or avidine-rhodamine (Vector Laboratories).

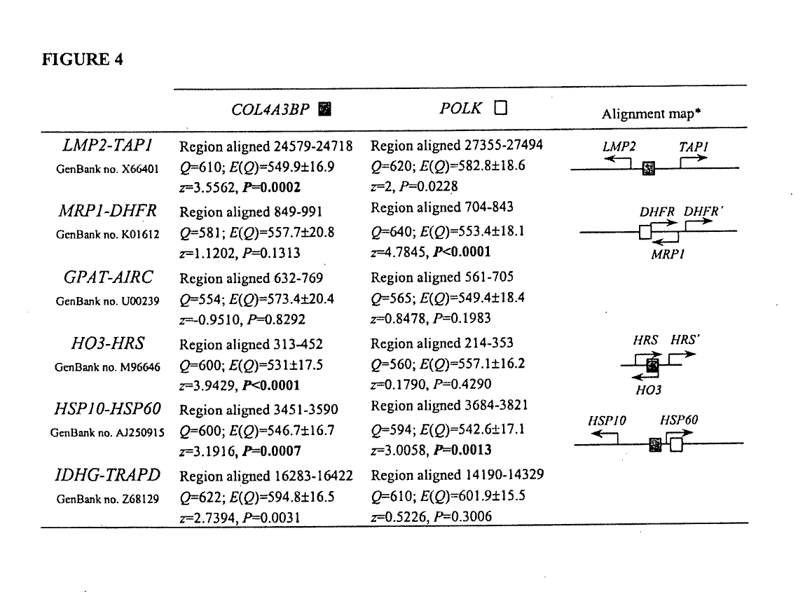

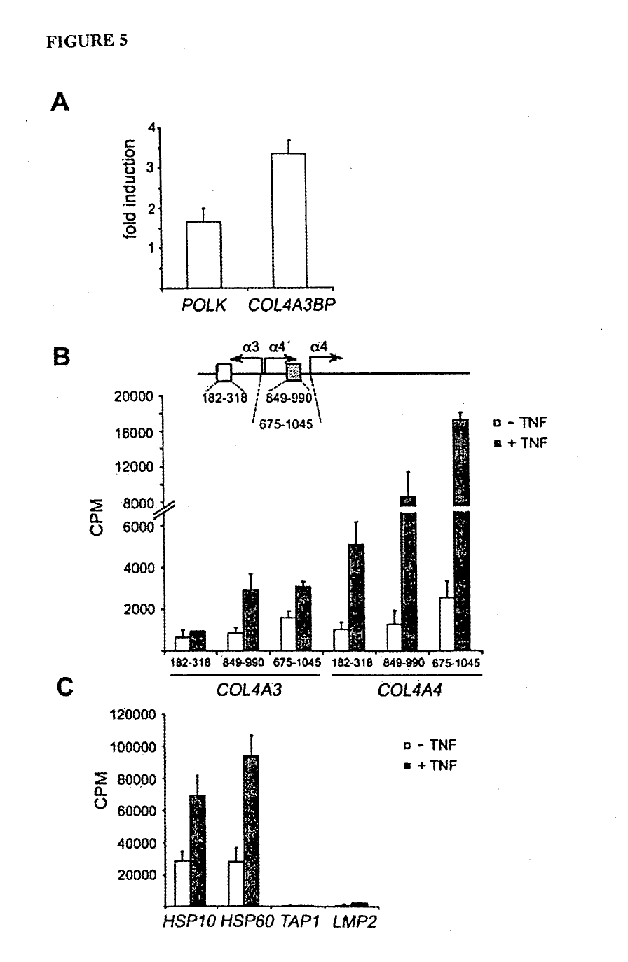

Computer analysis. Alignments were generated with the program GAP of the GCG-package (Genetics Computer Group). GAP uses the algorithm of Needleman and Wunsch [14] As originally introduced the algorithm sought to maximize a similarity, or quality (Q), between two sequences. From any pair of bases, an alignment can be extended in three ways: adding a base in each sequence, with a specified addition to the distance if the bases do not match, or adding a base in one sequence but a gap in the other, or vice versa. Introduction of a gap also contributes a specific amount to the distance. Formally, the best alignment will be the one that keeps up the relationship Q=max(x−Σzₖwₖ), where x is the number of matched pairs, zₖ the number of gaps with length k, and wₖ the penalty for a gap of length k. Many systems of gap penalty have been used; the liner system being the most commonly used because it saves computer time. In this system wₖ=α+βk, where a (the gap-opening penalty) and β (the gap-extension penalty) are non-negative parameters. Which alignment is preferable depends upon the penalty weights used. For example, a small a along with a big β will favor an alignment with many short gaps, whereas a large a with a small β will favor an alignment with few long gaps. The gap parameters employed in the analysis were α=50 and β=3. The statistical distribution of Q is not well characterized. Therefore, to assess the statistical significance of an alignment it is necessary to use a bootstrapping technique. In brief, the sequence being aligned is shuffled 100 times, maintaining its length and composition, and then realigned to the target POLK/COL4A3BP sequence. The average alignment quality, E(Q), plus or minus the standard deviation, of all randomized alignments can be used to evaluate the significance of the alignment. If the observed Q is significantly larger than that expected by chance, E(Q), then a P<0.05 would be obtained. FIGS. 4 and 5 show the observed Q values as well as E(Q) (±standard error).

Animal studies. The implication of TNF and GPBP in the development of murine systemic lupus erythematosus (SLE) was analyzed in F1 hybrids between NZW females and C57BL/6 (B6) males that over-express a human Bcl-2 transgene in the B cell compartment under the regulation of the SV40 promotor and IgM enhancer. These Bcl-2-transgenic F1 mice develop an aggressive SLE characterized by the production of a large spectrum of pathogenic autoantibodies resulting in the development of an immunocomplex-mediated gomerulonephritis and early death (50% of mortality is observed at 9-10 months of age) [15]. In contrast, non-transgenic (NZW×B6)F1 mice are immunologically normal and are used as controls. The development of the disease in the Bcl-2-transgenic F1 mice is believed to be a consequence of an over-expression of human Bcl-2 in B cells that prolongs the survival of potentially autoreactive B cells generated either in the bone marrow or in the germinal centers of secondary lymphoid organs in the course of T cell-dependent antibody responses, and also because of the genetic predisposition to SLE provided by the NZW genetic background. In this respect, several genetic loci associated with the production of autoantibodies and/or glomerulonephritis (GN) have been mapped in the NZW mouse strain. However, the nature of these genetic defects associated with the different autoimmune traits remains at the present largely unknown. The production of autoantibodies in Bcl-2-transgenic F1 mice is first observed at 2 months, and glomerular lesions are already evident at 3-5 months of age. As observed in other murine models of spontaneous SLE, both autoantibody production and GN are inhibited after the treatment from birth of (NZW×B6)F1-Bcl-2 mice with an anti-CD4 monoclonal antibody, indicating that the disease is a CD4-dependent phenomenon.

For some purposes, (NZW×B6)F1 mice were treated from birth with anti-CD4 antibodies as previously reported [16], and the presence of the transgene (Tg) in each animal determined as described [17]. The anti-CD4 treatment was continued for the F1Tg(+) up to three month and then half of mice were maintained without additional treatment whereas the other half were enrolled in a program with anti-TNF antibodies (V1q) essentially as described [18] but using 30 μl of V1q ascites three times per week. After two and a half months both anti-TNF treated and non-treated animals were sacrificed and one of the kidneys used for histology and immohistochemistry, and the other for mRNA studies. For similar purposes we also obtained the kidneys of animals representing the parental strands, female NZW and male C57BL/6-Bcl-2 and three month old (NZW×B6)F1Tg(−) and (NZW×B6)F1Tg(+) maintained without anti-CD4 treatment.

For other purposes, B6 mice were intraperitoneally injected with 50 μg of lipopolysaccharides (LPS) obtained from Salmonella minnesota (Sigma), which induces a dramatic increase in the serum levels of TNFα, resulting in the development of endotoxic shock [19]. Either three or six hours after LPS injection, mice were sacrificed and their kidneys immediately extracted, frozen in dry ice, and used for RNA isolation. Non injected C57BL/6 mice were similarly sacrificed and their kidneys obtained for use as controls.

Immunochemical techniques. Immunihistochemical studies were performed on formalin-fixed, paraffin-embedded mouse kidneys essentially as described [2,3], using GPBP polyclonal antibodies [2] at 1:50 dilutions. Prior to antibody detection, antigen retrieval was achieved heating with autoclave (1.5 atmospheres for 3 minutes in 10 mM sodium citrate buffer pH 6.0).

For some purposes the presence of anti-ssDNA autoantibodies was determined in the sera of the mice using an ELISA approach [17].

Results

Structural characterization of the 5′ region of COL4A3BP. To characterize the promoter region of COL4A3BP we first attempted to determine the transcriptional start site by primer extension analysis. However, and likely due to the high G+C content at the 5′-end untranslatable region (UTR)[2], we obtained premature stops during reverse transcription at positions 56, 61 or 68 of the cDNA in n4′ (GenBank accession no AF136450) (not shown). A similar negative results were obtained when a 5′-RACE approach was used to identify mRNA species extending beyond the 5′ end of n4′ (not shown). To overcome this inconvenient, we isolated and characterized by partial nucleotide sequencing ˜1.5 kb of genomic DNA located upstream of the 5′-UTR of n4′, and screened a cDNA human library to identify clones containing additional 5′-UTR of GPBP not present in n4′. We isolated and sequenced 1.3-kb HeLa 4.1 ((SEQ ID NO:2) GenBank accession no AF315601), which did not overlap with n4′ although contained sequence present in the 1.5-kb DNA. Because HeLa 4.1 did not contain open readings of consideration in the six frames (not shown), its cDNA likely represents either 5′-UTR of GPBP not present in n4′ or sequence corresponding to an UTR of other gene mapping 5′ of COL4A3BP. The first possibility was abandoned since we failed to amplify by RT-PCR a continuous cDNA fragment containing both HeLa 4.1 and n4′ sequences (not shown). As expected, however, we succeeded obtaining a DNA fragment of 955-bp ((SEQ ID NO:3) GenBank accession no AF315603) when subjecting human DNA to PCR using ON-GPBP-18m, a forward primer derived from HeLa 4.1, and ON-GPBP-6c, a reverse primer derived from n4′ (FIG. 1), thus supporting the second possibility. To assign a gene for HeLa 4.1, we first search at the data banks and we found not a gene to contain HeLa 4.1 cDNA sequence. However, when we included in the search the 418-bp DNA connecting HeLa 4.1 and n4′ sequences at the human genome which is comprised in SEQ ID NO:3 (FIG. 1), we found that it contained inverted 159-bp of 5′-UTR present in the mRNA encoding for pol κ (GenBank accession no AF163570), a novel member of the growing family of DNA polymerases that display ability to bypass mismatches during DNA replication [5). This suggested that HeLa 4.1 contained part of the 5 ′UTR of pol κ not present in the mRNA molecular species previously characterized. Therefore HeLa 4.1 represented either an alternatively spliced variant or an alternative transcriptional start site. Using a RT-PCR approach we have not been able to identify a mRNA species containing both HeLa 4.1 and the 159-bp exon sequence (not shown), suggesting that HeLa 4.1 likely represents an alternative transcription start site. Nevertheless to assess that HeLa 4.1 indeed contains 5′-UTR of POLK we have performed specific PCR on human muscle cDNA and identified a molecular species containing both HeLa 4.1 and pol κ coding sequence (GenBank accession no AF318313). The resulting cDNA fragment, however, did not contain the full HeLa 4.1 sequence and contained 142-bp of UTR not present neither in HeLa 4.1 neither in the original pol K sequence reported [5], thus confirming the existence of at least three mRNA species for pol K with different 5′-UTR and suggesting that the 140-bp flanked by the most 5′-UTR of the two genes (FIG. 1) (SEQ ID NO: 6 and SEQ ID NO:7) (SEQ ID NO:33 and SEQ ID NO:34 show the corresponding mouse 140 bp sequence) contains a bidirectional promoter. Finally, we have used RNA-protection assays to map the transcriptional start sites for each of the genes. When radiolabeled RNA probes representing the antisense strand of POLK or COL4A3BP between the ApaI and EclXI sites (FIG. 1) were separately hybridized with human RNA, one major fragment of 169 and 63 nucleotides long was respectively protected from RNase digestion. Minor fragments, one of 151 nucleotides for POLK and several others for COL4A3BP were also protected (not shown). However, from the comparison of DNA and cDNA sequences the fragments expected to be protected by the exon 1 were 159 and 55 nucleotides long respectively. Therefore, these results would suggest the existence of two major transcriptional start sites one for POLK and another for COL4A3BP which extend the 5′ end of the corresponding mRNAs ten and eight nucleotides into the intergene region with respect to the cDNA sequence previously reported (FIG. 1). The significance of the additional protected fragments identified is uncertain as may represent alternative transcriptional start sites, a common feature in bidirectional promoters [20-22] or alternatively, and because of the high content in G+C, lack of protection of the more abundant fragments due to defective pairing caused by secondary structures. Nevertheless these findings suggest that the genomic region flanked by the two major transcriptional start sites contains the structural requirements for bidirectional transcription. In this respect the size, the presence of alternative transcriptional start sites, a Sp1 site, a single TATA box and the high content in G+C are structural features shared by other bidirectional promoters [20-22].

Chromosomal mapping of the human COL4A3BP gene. By FISH analysis others have shown a single locus for POLK at band 5q13 [5]. In similar studies and consistent with the proposed head-to-head arrangement of COL4A3BP and POLK, two independent overlapping DNA fragments of COL4A3BP hybridized with a single locus mapping at 5q12-13. According to the last publicly available data on the human genome sequence, both COL4A3BP and POLK map to 5q13.3. In the last freeze of the sequence (http://genome.ucsc.edu/goldenPath/apr2001Tracks.html) there still remains a gap between both genes that is bridged with the sequence reported here (SEQ ID NO:3) GenBank accession no AF315603) (FIG. 1). Finally whereas this manuscript was being completed a GenBank accession number AB036934 was released which contained the sequence reported here thus confirming the head-to-head arrangement we have proposed.

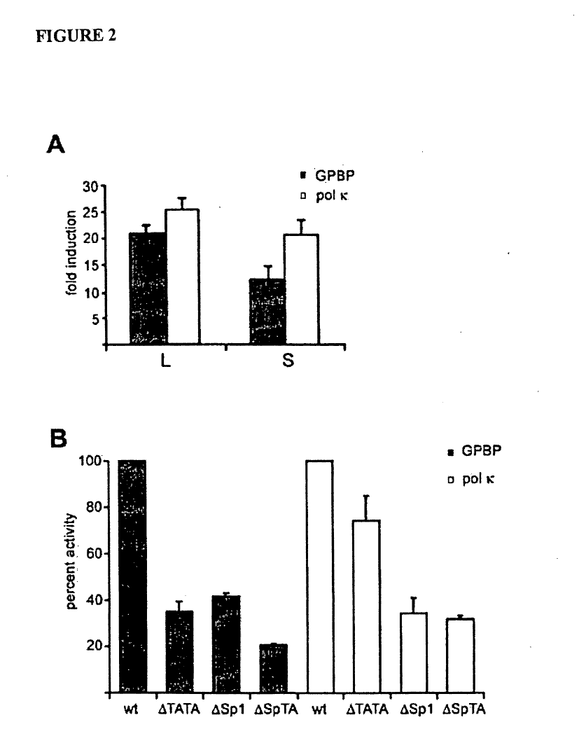

Characterization of the bidirectional transcription unit for POLK and COL4A3BP. To investigate the presence of a bidirectional promoter in the intergene region we cloned in pΦGH each of the two orientations of a 772-bp DNA fragment (SEQ ID NO: 4 and SEQ ID NO:5) encompassing the region of interest (LpromPolκ and LpromGPBP) and we assessed their ability to drive heterologous gene expression in NIH 3T3 cells (FIG. 2A). The 772-bp fragment efficiently promoted heterologous gene expression in each orientation, 25-fold over control in the POLK direction for 21-fold in the COL4A3BP orientation. When we assessed the transcriptional activity of the 140-bp DNA region (shaded sequence in FIG. 1) containing the identified 5′ transcriptional start sites for each gene (SEQ ID NO:6 and SEQ ID NO:7) (SpromPolκ and SpromGPBP), we observed a reduction in the activity that was more evident for COL4A3BP orientation than for PdLK, a 45% reduction versus 18%, indicating that although the 140-bp contains the core of the bidirectional transcriptional unit and the structural requirements for divergent transcription, in the flanking structural gene regions there are regulatory elements that modulate both gross activity and relative transcription rates in each orientation. In this regard in the exon 1 of POLK there is a Sp1 site (FIG. 1) that could account at least in part for the higher transcriptional activity of the larger promoter constructs.

The contribution that the individual DNA elements identified in the 140-bp DNA region had on the transcriptional activity was assessed using promoter constructs in which the Sp1 site or/and the TATA box were deleted (FIG. 2B). The removal of each of the two DNA elements had consequences in the transcriptional activity of the promoter although these were significantly different for each orientation. Thus Sp1 site deletion greatly impaired transcription in the two orientations although this was more evident for POLK transcription. In contrast TATA box deletion greatly reduced transcription in COL4A3BP direction but had little effect over POLK transcription. Finally, double deletions were additive in the negative effects over transcription in either orientation reaching values slightly above those obtained with empty vector (7-12%). These results suggest that the TATA box is mainly used for COL4A3BP expression whereas Spl is the major element through which operates the bidirectional expression.

The expression of the bidirectional unit in human tissues The transcriptional activity of the bidirectional promoter in human tissues was investigated by Northern blot analysis. With the exception of brain and pancreas that showed a relatively reduced expression of pol κ, comparison of mRNA levels among tissues revealed that the two genes are expressed in a coordinated manner in normal human tissues, whereas coordination appears to be disrupted during cell transformation as comparison of mRNA levels in human cancer cell lines showed that cells with a relative higher expression of GPBP expressed relatively less pol κ and vice versa (not shown). In either case this suggests that pol κ and GPBP are likely partners in specific biological functions and that the head-to-head arrangement of the corresponding genes is the strategy to co-regulate their expression.

Sequence homology between POLK/COL4A3BP and COL4A3/COL4A4 promoters. Several housekeeping genes, including those encoding α chains of collagen type IV, are transcribed from short, bi-directional, G+C rich promoters containing Sp1 sites [22]. Six related genes organized in three transcriptional units encode the human α(IV)chains (α1/α2, α3/α4 and α5/α6) [23-25] which likely have evolved from a primitive genetic unit the proto-α1/proto-α2 resulting from duplication and inversion of a unique primitive gene with an unidirectional promoter [26-29]. Consistent with this evolutionary model the structural genes for α1,α3 and α5 on one site and α2,α4 and α6 on the other, are more closely related [26-29].

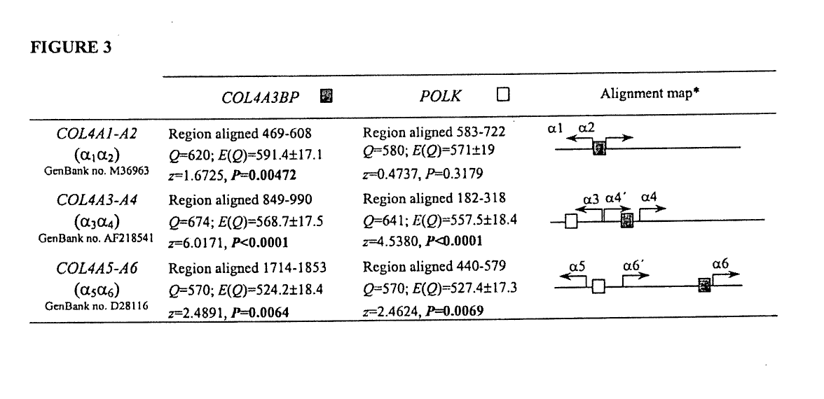

Because GPBP has been shown to bind and phosphorylate the α3(IV)NC1 domain and a similar binding to the homologous α1 and α5 NC1 domains has been found to exist [3] we searched for sequence homology between the 140-bp of POLK/COL4A3BP containing the intergene region and genomic regions expected to contain the core of each transcriptional collagen IV unit (FIG. 3). The COL4A3/COL4A4 junction (GenBank accession no AF218541) contains regions conspicuously homologous to each of the two orientations of the 140-bp yielding alignments with a high statistical significance (P<0.0001). One of the alignments (SEQ ID NO: 10 (A3 orientation) and SEQ ID NO:11 (A4 orientation) maps between the transcriptional start site of COL4A3 and one of the two alternative transcriptional start sites of COL4A4, whereas the other (SEQ ID NO: 8 (A3 orientation) and SEQ ID NO:9 (A4 orientation) is at the first intron of COL4A3 upstream of the second transcriptional start site for COL4A4. Similarly, each orientation of the 140-bp was homologous to DNA regions in the COL4A5/COL4A6 junction (GenBank accession no D28116) with alignments also highly significant (FIG. 3). One of the aligned regions (SEQ ID NO: 18 (A5 orientation) and SEQ ID NO:19 (A6 orientation) maps in between the two structural genes at the intergene region flanked by the transcriptional start site for COL4A5 and one of the two alternative transcription start sites for COL4A6, whereas the other (SEQ ID NO:20 (A5 orientation) and SEQ ID NO:21 (A6 orientation) is located upstream of the second transcription start site of COL4A6. Finally, only one region (SEQ ID NO:22 (A1 orientation) and SEQ ID NO:23 (A2 orientation)of COL4A1/COL4A2 junction (GenBank accession no M36963) aligned significantly with the orientation of the 140-bp expressing COL4A3BP (FIG. 3). Interestingly no alternative transcription start sites for COL4A2 have been reported. Although the values for Q and E(Q) in the alignment with COL4A1/COL4A2 compromises its biological significance, the preferred alignment of the 140-bp at a 127-bp region between the two 5′-UTR in COL4A1/COL4A2, in a search of 2184-bp of COL4A1/COL4A2 nucleotides, suggests that the homology is of biological significance.

Sequence homology between COL4A3BP/POLK and other bidirectional human promoters. The genomic regions representing the intergene and flanking structural genes of a number of bidirectional transcriptional units others than collagen α(IV) (GenBank accession no X66401, K01612, U00239, M96646, AJ250915 and Z68129) [30-37] were similarly analyzed for sequence homologies with the 140-bp of POLK/COL4A3BP (FIG. 4). Four out of six transcriptional units yielded statistically significant alignments at the intergene region where the corresponding core promoter is expected to map. These were LMP2/TAP1; MRP1/DHFR; HO3/HRS and HSP10/HSP60 respectively encoding low molecular mass polypeptide 2 and transporter associated with antigen processing 1; mismatch repair protein 1 and dihydrofolate reductase; histidyl-tRNA synthetase homolog and histidyl-tRNA synthetase; and, mitochondrial heat shock protein 10 and heat shock protein 60. The most remarkable alignments were those resulting from the comparison of the promoter sequence representing the orientation for COL4A3BP transcription with LMP2/TAP1 or HSP10/HSP60 transcriptional units. In the first case, among 66061-bp containing five structural genes of the MHC class II and the corresponding intergene regions the preferred alignment was in the ˜600-bp at the intergene region of LMP2/TAP1 unit with a probability of 0.0002 that the homology could be found by chance. In the second case, a similar result was obtained when the search for sequence homology was done over 16986-bp which contained the two structural genes and ˜550-bp of intergene region. Finally, the promoter sequence representing the orientation for POLK transcription aligned most significantly (P<0.0001) with the MRP1/DHFR junction region immediately upstream (nucleotides 704-843) (SEQ ID NO: 16 (MRP1 orientation) and SEQ ID NO:17 (DHFR orientation)) of the first transcription start site for DHFR (nucleotide 844). It is also of interest to mention the statistical significance of the alignment between the transcription orientation for COL4A3BP and POLK with the first exon of HO3 and HSP60 (P<0.0001 and P=0.0013) respectively. In the case of HO3 (SEQ ID NO: 24 (HO3 orientation) and SEQ ID NO:25 (HRS orientation)), the alignment maps upstream of an alternative transcriptional start site for HRS (HRS'). Other alignments were either marginally significant and/or mapp d at regions unlikely to contain a bidirectional promoter e.g. COL4A3BP orientation alignment with IDHG-TRAPD (FIG. 4).

These data demonstrate that the COL4A3BP/POLK base pair promoter sequence, which was shown to comprise a bi-directional promoter, contain sequences that are significantly homologous to a number of other known bi-directional promoters, and thus probably constitute regulatory elements shared in common by a family of bi-directional promoters.

TNF induce the POLK/COL4A3BP and COL4A3/COL4A4 promoters in transient gene expression assays. GPBP is highly expressed in apoptotic blebs in tissues undergoing autoimmune attack and is virtually not expressed in transformed cell lines [3]. Consequently to identify modulators of the transcriptional activity of POLK/COL4A3BP, a number of cytokines (TNFα, TNFβ and γIFN) with ability to cause cell death, with an anti-tumoral potential and with a role in the immune defense but also in autoimmune pathogenesis were used as inducers on cultured NIH3T3 or HeLa cells transfected with the 140-bp promoter constructs (SpromPolκ and SpromGPBP). Whereas we found no effect on the transcriptional activity of the constructs when inducing the cells with IFNγ (20 ng/ml) or when inducing HeLa cells with any of the three cytokines, we found that either TNFα (10 ng/ml) or TNFβ (50 ng/ml) induced the two promoter constructs in NIH 3T3 cells (FIG. 5A), however, the induction from the 140-bp promoter was more efficient in the COL4A3BP than in the POLK direction.

To date no functional characterization of the transcriptional unit for COL4A3/COL4A4 has been reported. To explore the biological significance of sequence homology between this bidirectional promoter and the promoter of POLK/COL4A3BP (SEQ ID NOS: 6-7), we cloned each of the two orientations of the COL4A3/COL4A4 homologous regions (FIG. 3) (SEQ ID NOS:8-11) in pΦGH vector and assessed transcriptional activity in NIH3T3 cells in response to TNF (FIG. 5B). No transcriptional activity was observed in the absence of TNF treatment for any of the four constructs indicating that differently to the POLK/COL4A3BP promoter (FIG. 2) the two homologous regions in COL4A3/COL4A4 do not show constitutive transcriptional activity in NIH 3T3 cells. In contrast, when the cells were induced with TNF the two DNA regions were able to drive reporter gene expression although more efficiently for COL4A4 than for COL4A3 direction. In fact the later was only appreciable when assaying the promoter mapping at the intergene region (nucleotides 849-990 of AF218541) (SEQ ID NO:10), whereas the promoter mapping inside the COL4A3 (nucleotides 182-318 of AF218541) (SEQ ID NO:8) showed no inducible activity in this direction. In order to further support the bidirectional activity of the 849-990 region the entire intergene region flanked by the two transcriptional start sites (nucleotides 675-1045) (SEQ ID NOS:12-13) was similarly cloned and assayed. As observed for the 849-990 constructs these had not significant constitutive transcriptional activity and showed a limited response to TNF in COL4A3 direction that contrasted with the induction of the transcriptional activity in the COL4A4 direction which resulted to be significantly higher than when assaying the 849-990 construct. These results suggest the existence of two independent promoters in the DNA region that connects the 5′ ends of COL4A3 and COL4A4 which respond to TNF, one bidirectional and another unidirectional. The low activity of the bidirectional promoter in the COL4A3 direction may be due to the existence of regulatory elements far apart from the core or to the lack of specific transacting factors in NIH 3T3. In any event these results suggest that the POLK/COL4A3BP and the COL4A3/COL4A4 bi-directional promoter are coordinately regulated by TNF, and verify the biological significance of the homology found between the POLK/COL4A3BP 140 base pair bi-directional promoter fragment, and the homologous promoter fragments from the COL4A3/COL4A4 promoter.

TNF induce dual homologous bidirectional promoters other than COL4A3/COL4A4. The coordinated regulation above could be understood as a part of a regulatory mechanism which depend of TNF in the context of the previously identified biological partnership of GPBP and the α chains of collagen IV [2,3], however, no immediate biological relation exists between pol κ and GPBP, and between GPBP and the products of the other bidirectional units which have been identified by sequence homology. To explore the scope of our findings we cloned and similarly assayed the 140-bp homologous DNA fragment mapping at the intergene region of LMP2/TAP1 (SEQ ID NO: 14 (LMP2 orientation) and SEQ ID NO:15 (TAP1 orientation) and HSP10/HSP60 (SEQ ID NO: 26 (HSP10 orientation) and SEQ ID NO:27 (HSP60 orientation), which represented the statistically more significant homologies (FIG. 4). Transient gene expression assays carried in NIH 3T3 cells show that whereas no transcriptional activity was found in any of the two orientation of the LMP2/TAP1 fragment (nucleotides 24579-24718 of X66401) (SEQ ID NOS: 14-15) the fragment of HSP10/HSP60 (nucleotides 3451-3590 of AJ250915) (SEQ ID NOS: 26-27) displayed both constitutive and inducible activity which was similar for each of the two orientations (FIG. 5C). Previous studies have shown that the LMP2/TAP1 unit responds to TNF and that the major transcriptional start and regulatory sites for either the two orientations in response to this cytokine mapped at the TAP1-proximal region (nucleotides 24757-24965 of X66401) [35]. However in this study the ability of this particular fragment to transcribe LMP2 in response to TNF was not assayed and therefore no direct experimental evidence was provided to rule out that the DNA region containing the homologous 140-bp indeed does not contain TNF responsive elements for LMP2 transcription, moreover, when the site at the TAP1-proximal region accounts only for the 65% of the total induction in this direction.

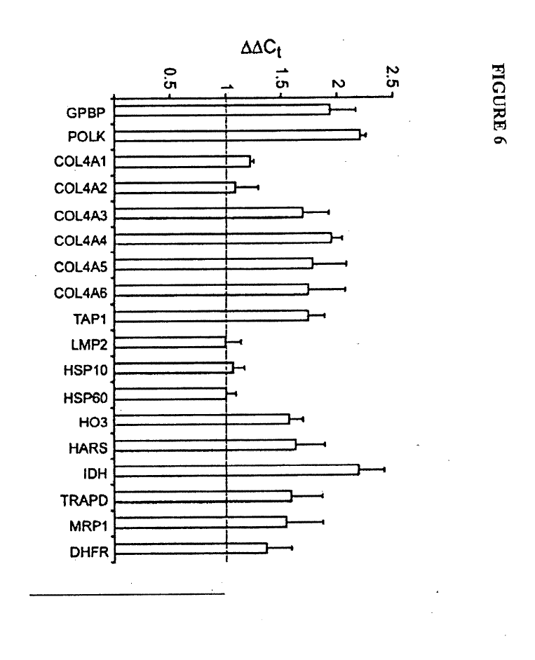

Finally the transcriptional induction of the different dual units in response to TNF was investigated in cultured human hTERT-RPE1 cells by determining mRNA levels using a Real Time PCR approach (FIG. 6). Since these cells are immortalized by over-expression of telomerase, they can be considered as primary cells, and thus more physiologically relevant than established cell lines. We have determined that these cells produce α3(IV) and GPBP. Furthermore, they are derived from retina, and retinal basement membrane contains abundant α3-α4-α5 collagen IV chains, and similarly to glomerular basement membrane it has been shown to contain linear deposits of autoantibodies in Goodpasture patients. In these cells TNF induced the transcription of POLK and COL4A3BP however when we assessed the level of expression of GPBP and GPBPΔ26, the two alternatively spliced products of COL4A3BP, we found that the induction depended mainly of GPBP and little induction of GPBPΔ26 was observed (not shown). The effects on the transcriptional units for the α chains of collagen IV genes varied, thus the promoter for the ubiquitous α1 and α2 chains, which displayed the less significant homology, was not inducible whereas the promoters for the α3-α6 chains with a more restricted tissue distribution and displaying the most significant alignments were induced to a similar extent and in the two transcriptional directions. The studies on dual units coding for proteins other than collagen IV α chains revealed that LMP2/TAP1 unit responded to TNF although the induction was only detected in the TAP1 direction whereas no induction of the promoter for HSP10/HSP60 was detectable in these cells. Interestingly the rest of the bidirectional units that the computer analysis showed to contain 140-bp homologous regions also were inducible by the cytokine including IDHG/TRAPD which homologous region mapped ˜1.5 kb 3′ of the polyadenylation signal of TRAPD. The coordinated expression of IDHG/TRAPD and POLK/COL4A3BP was also evident when the expression in different human tissues of GPBP and IDHγ was compared using standardized Northern blots (compare FIGS. 2 of Ref. 2 and Ref. 37).

All these data indicate that at least for the number of genes we have reported the head-to-head arrangement is a convergent evolution phenomenon to coordinate their expression in response to TNF and that the 140-bp homologous modules contain responsive elements for the coordinated expression. Finally, our findings indicate that TNF not only induces the expression of COL4A3BP by increasing the copy number of the corresponding mRNA molecular species but also increases the relative expression of GPBP versus GPBPΔ26, a phenomenon which we have previously shown to be related with autoimmune pathogenesis [3].

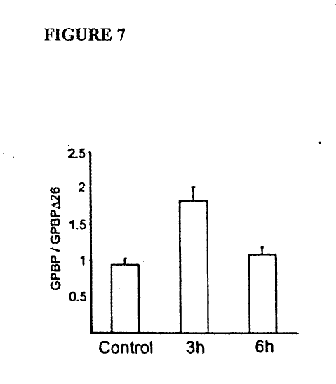

Evidences for TNF increasing the relative expression of GPBP in vivo, a phenomenon critical for SLE development in a lupus prone mouse model. The role of TNF regulating GPBP/GPBPΔ26 ratio in the kidney was explored in B6 mice by inducing endogenous TNF production in response to LPS (FIG. 7). At the time of injection the GPBP/GPBPΔ26 values were below 1, however after three hours of LPS injection the GPBP/GPBPΔ26 ratio reached values of 2 to finally return to near initial values after six hours of LPS injection. Contrary to what we have found when inducing hTERT-RPE1 cells the total copy number of these mRNA species with respect to the copy number of mRNA for GAPDH did not varied significantly (not shown), thus indicating that the relative increase of GPBP at the three hours was a consequence of a reduced expression levels of GPBPΔ26.

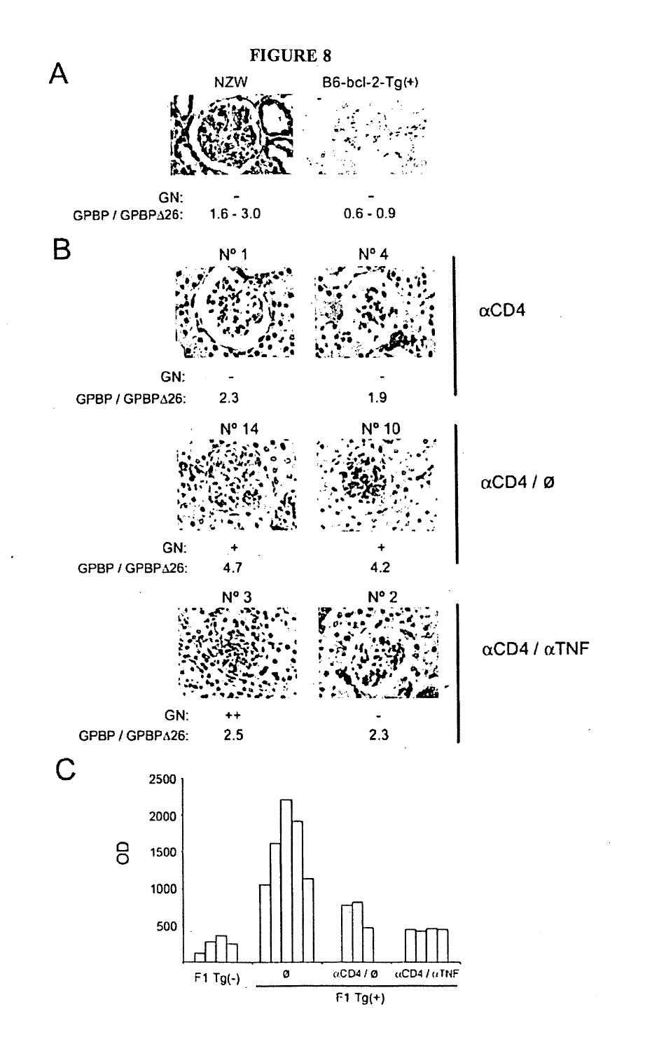

To explore the role of TNF inducing the expression of GPBP in an autoimmune response we first determined the expression of GPBP and GPBPΔ26 in a recently reported lupus prone model [15] which we have described here under Material and Methods (FIG. 8A). In this model the genetic background that predisposes female NZW to undergo SLE is “activated” by transgenic over-expression of Bcl-2 in the B cells compartment in the F1 generation which develops a severe autoimmune GN that is evident at the third month of life. We have previously reported that GPBP is poorly expressed in the kidney of Balb/c mice and that glomerular expression of GPBP was not detectable by standard immunochemical techniques [3]. Consistently we have not detected expression of GPBP in the glomerulus of the C57BL/6 (B6) male which over-express Bcl-2 transgene and we have found that in these kidneys the levels of mRNA for GPBP were lower than for GPBPΔ26 (GPBP/GPBΔ26<1). In contrast, the kidney of a NZW female expressed GPBP to a higher levels than GPBPΔ26 (GPBP/GPBPΔ26 values between 1.6 and 3.0) and contained hyaline deposits in the glomerulus which were detectable by standard immunochemical techniques using GPBP-specific antibodies. Finally, we found that in the (NZW×B6)F1 generation, and with independence of Bcl-2 transgene (Tg) expression, the GPBP/GPBPΔ26 values in the kidney were higher than in NZW (GPBP/GPBPΔ26>3.0) and showed important variations between homologous animals (GPBP/GPBPΔ26 values ranged between 3.2 and 15.5). The relative increase of GPBP however did not represent in any case (NZW or F1) an absolute increase in the mRNA copy number of GPBP which was always 5-15% of the mRNA copy number of GAPDH but rather was caused by a decrease in the expression of GPBPΔ26 (not shown). Immunohistochemical studies showed that both (NZW×B6)F1Tg(+) as well as (NZW×B6)F1Tg(−) did not express GPBP-containing hyaline deposits at the glomerulus and only the (NZW×B6)F1Tg(+) developed an autoimmune glonerulonephritis (not shown).

Treatment with anti-CD4 immediately after birth (see Material and Methods) had important consequences in both mRNA expression and immunohistochemical pattern of the (NZW×B6)F1Tg(+) (FIG. 8B). Thus the GPBP/GPBPΔ26 ratio was substantially reduced with respect to untreated animals and dropped to levels similar to those of NZW and the expression of GPBP at the glomerulus as estimated by immunohistochemistry was greatly reduced in comparison with NZW. Finally, interruption of anti-CD4 treatment for two and a half months resulted in an increase in the relative expression of GPBP in the kidney (GPBP/GPBPD26>4.0) and in the restoration of specific GPBP deposits at the glomerulus unless anti-TNF antibodies were administered, in which case the ratio GPBP/GPBPΔ26 remained down and the presence of GPBP-conataining deposits at the glomerulus was not detectable by immunohistochemical techniques (FIG. 8B). Histological evaluation of the kidneys revealed that as expected early treatment with anti-CD4 prevented development of GN whereas interruption of this treatment resulted in a progressive restoration of the GN unless the anti-TNF program was started in which case the consequences were unequal, one mouse did not developed GN whereas the other showed a more severe nephritis.

To investigate the consequences that the immunological treatment had on the autoimmune response the levels of anti-ssDNA autoantibodies in the sera (a standard and very sensitive marker for autoimmunity) of six month old (NZW×B6)F1Tg(−) or (NZW×B6)F1Tg(+) maintained untreated, were compared with the levels of these autoantibodies in (NZW×B6)F1Tg(+) treated with anti-CD4 for three months and either untreated or treated with anti-TNF for three additional months (FIG. 8C). As expected (NZW×B6)F1Tg(−) showed levels of autoantibodies in the background range (0.1-0.5) whereas untreated (NZW×B6)F1Tg(+) showed elevated titers of autoantibodies (1.0-2.2 OD). Treatment of the (NZW×B6)F1Tg(+) for three months with anti-CD4 and further maintained with anti-TNF up to six months efficiently inhibited the autoimmune response as estimated by the maintenance of autoantibodies level at the background range. In contrast the (NZW×B6)F1Tg(+) which were kept untreated for three months after the anti-CD4 treatment displayed autoantibodies values in between the untreated and the anti-TNF treated suggesting that the autoimmune response starts as the T cell population increases, unless anti-TNF is added, in which case the autoimmune response remains silent.

From all these data we conclude that the autoimmune response in the lupus prone model studied is mediated by TNF and operates through an elevated ratio of GPBP/GPBPΔ26.

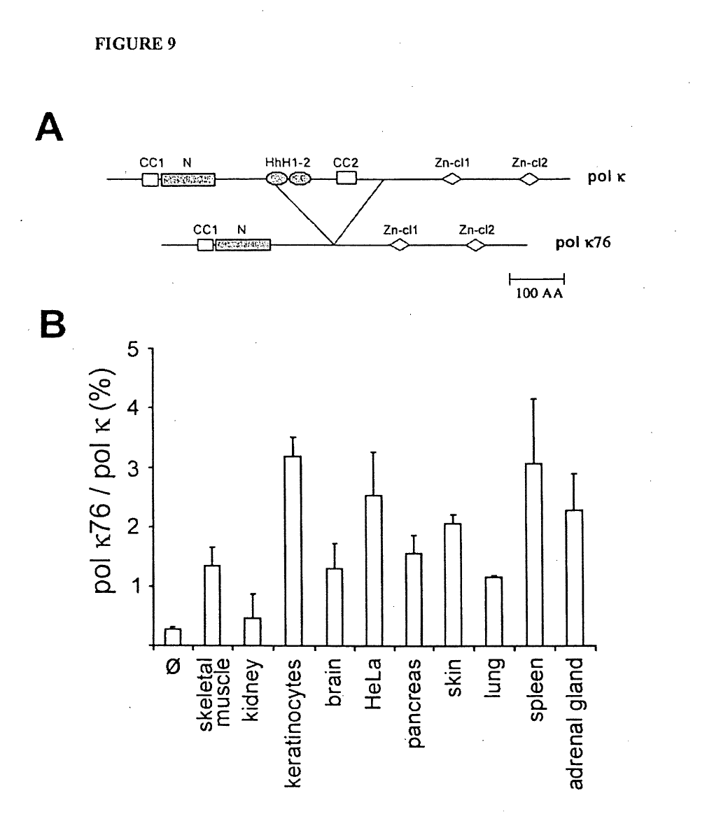

Molecular cloning of a 76-kDa alternatively spliced variant of DNA polymerase κ. Alternatively spliced variants of pol κ have been reported to exist in human and mouse testis [5]. The presence in HeLa and in human striated muscle of molecular species with different 5′-UTR (see above) also indicated the presence of molecular species representing alternatively spliced variants previously unrecognized. We have use RT-PCR on total human RNA from foreskin and we have cloned a previously unidentified mRNA species for pol κ. This novel mRNA species contain a 672-residue open reading frame predicting pol κ76, a 76-kDa pol κ isoform (GenBank accession no AF315602) (SEQ ID NO:31), which represents an alternatively exon splicing variant that diverged with respect to the alternatively spliced isoforms previously identified in that exon skipping does not cause a reading frame shift but eliminates the bulk of the sequence predicting two in tandem helix-hairpin-helix domains and a coiled-coil motif characteristic of the primary product (FIG. 9A).

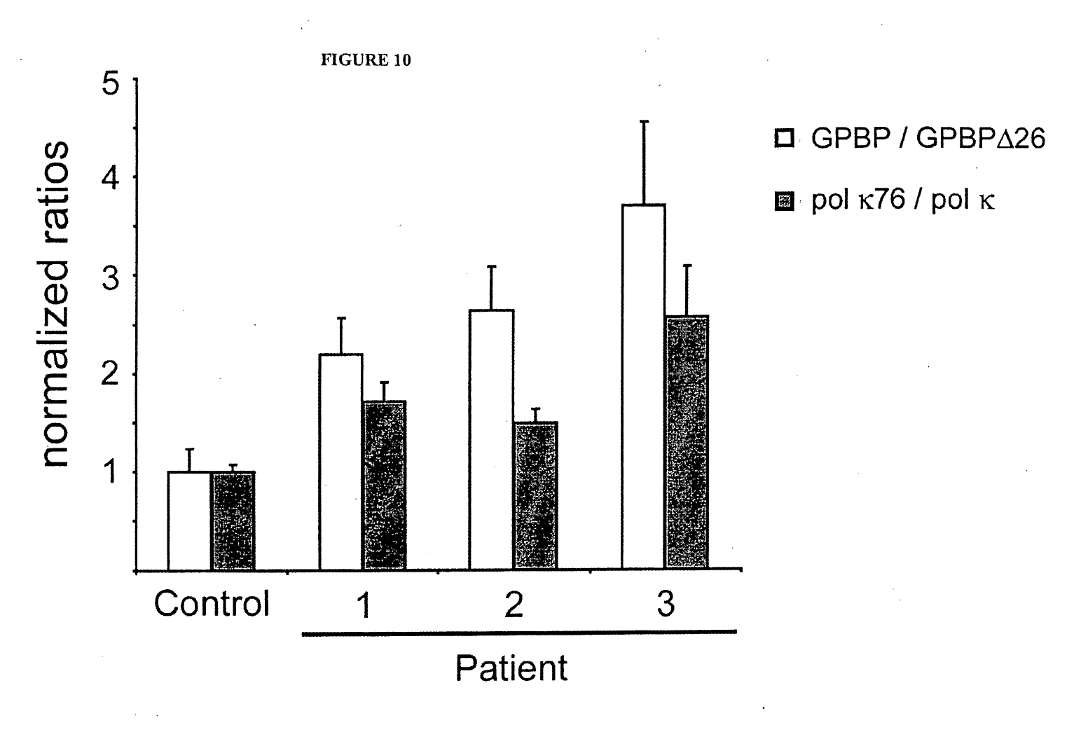

To estimate the relative expression of this novel molecular species in human tissues we performed specific Real Time PCR on several cDNA libraries or reverse transcriptase reactions from human tissues (FIG. 9B). Pol κ76 resulted to be a minor form which was comparatively more abundant in skin and in keratinocytes than in the rest of the tissues studied. The relative higher expression in the keratinocytes of the skin, a cell with an ongoing apoptotic program required for adequate maturation, prompted the idea that pol κ76 may be part of the cell machinery involved in the apoptotic program in which GPBP has been proposed to be involved in these cells [3]. We have investigated using a yeast two hybrid system the existence of protein-protein interactions between pol κ/pol κ76 and GPBP/GPBPΔ26 and we got no positive results (unpublished observations). However, we demonstrated that pol κ76 interacts with a protein that also interacts with GPBP/GPBPΔ26 (not shown). This data further suggests that GPBP and pol κ76 are partners in specific apoptotic pathways relevant in keratinocyte maturation and which become deregulated during autoimmune pathogenesis. We have previously reported that in the skin undergoing autoimmune attack there is a relative increase in the expression of GPBP with respect to GPBPΔ26 therefore resulting in increased values for the GPBP/GPBPΔ26 ratio [3], and suggesting that during pathogenesis changes in the exon splicing pattern of COL4A3BP also occur. In order to assess if this condition applies for POLK gene expression, affected skin from patients undergoing cutaneous lupus were individually RNA extracted and the mRNA levels for pol κ, pol κ76, GPBP and GPBPΔ26 measured. We have found that in these patients elevated pol κ76/pol κ ratios correlated with elevated ratios of GPBP/GPBPΔ26 (FIG. 10).

Discussion

In normal human tissues GPBP is expressed at a lower level than GPBPΔ26, an alternatively spliced variant devoid of 26-residues serine-rich motif which represents a less active isoform of the protein kinase [3]. Although GPBP and GPBPΔ26 are widely expressed in human tissues they show a preferential expression in cells and tissue structures which are the target of common autoimmune responses. [2,3]. These isoforms represent two different strategies to regulate the activity of a common catalytic domain, and several lines of evidence indicate that homeostasis is achieved by a balanced expression of each isoform, whereas a breakage of the homeostasis caused by a relative increase in GPBP expression results in autoimmune pathogenesis [3].

GPBP is expressed at very low levels in cancer cells and is highly expressed in apoptotic blebs of differenced keratinocytes at the periphery of normal epidermis [3]. Keratinocytes from patients suffering from skin autoimmune processes show an increased sensitivity to UV-induced apoptosis, and a premature apoptosis at the basal keratinocytes has been reported to occur in these patients [38-41]. Consistently, we have found GPBP to be expressed in apoptotic bodies expanding from basal to peripheral strata in epidermis undergoing an autoimmune attack [3]. Altered autoantigens including phosphorylated versions thereof have been reported to be produced and released from these apoptotic bodies [40]. All these suggest that GPBP is part of an apoptotic-mediated strategy for desired cell removal that generates aberrant counterparts of critical cell components and operates illegitimately during autoimmune pathogenesis [3].

It has been shown that dinB1 (pol IV) and the eukaryotic counterpart pol κ induces spontaneous mutation on undamaged DNA [4,6,7], likely as a result of a high error nucleotide incorporation rates and an efficient mismatch extension [7). The latter feature largely depends on the formation of a primer-template misalignment that generates −1 frameshift products [4,6].

The coordinated expression of COL4A3BP and POLK demonstrated herein suggest that the products encoded by these genes are partners in specific cell program(s), and that pol κ may represent a somatic mutation-based strategy to generate structural diversity which in some instances, such as in keratincocytes could be used to generate aberrant counterparts of critical cellular components as part of an apoptotic strategy. The disruption of the coordinated expression of the two genes during cell transformation (see Northern blot results) and its maintenance at higher levels in autoimmune affected tissues further supports the implication pol κ/κ76 in apoptotic strategies relevant in autoimmune pathogenesis. Finally, disruption of transcriptional coordination of POLK and COL4A3BP may be required in cancer to prevent cell death but also autoimmune attack during tumor growth.

Alternative exon splicing of the pre-mRNA of pol κ serves to generate three different types of mRNA products. Transcripts encoding truncated forms of the polymerase contain divergent, shortened C-termini that are devoid of the Zn clusters and bipartite nuclear localization signals [5], and therefore are expected to play a regulatory role in the expression or activity of the primary pol κ product rather than to represent an alternative replicating enzyme. Transcripts with alternative 5′-UTR, essentially differing from each other in the nucleotide sequence at the vicinity of the translation start site, may represent mRNAs translated with different efficiency or molecules with different stability.

Pol κ76 is the first member of the UmuC/DinB superfamily that contains the N-terminal nucleotidyl transferase domain, but lacks the helix-hairpin-helix motifs and the predictable coiled-coil structure at the C-terminal conserved domain. This isoform retains the Zn clusters for DNA binding also existing in other family members devoid of nucleotidyl transferase domain, but with demonstrated DNA repair activity (Rab18 and Snm1) [5]. The helix-hairpin-helix has been implicated in non-specific binding to DNA and the coiled-coil structure could mediate protein-protein interactions. The fact that pol κ76 still harbors the critical structural requirements for DNA polymerase, and also maintain those characteristic of the DNA repair related enzymes, suggest that pol κ76 may represent the version of pol κ to generate aberrant counterparts of critical cell components in the context of a common apoptotic-mediated strategy for a desired cell removal, similarly to the proposed role for GPBP versus GPBPΔ26 in keratinocyte apoptosis. [3]

Multiple sclerosis is an autoimmune disorder with a complex mode of inheritance. A genome search has suggested co-segregation of a locus for this disease with the marker D5S815 [42]. Whereas this marker maps at positions 79000 Kbp and 81556 Kbp from the telomere according to GeneMap (http://www.ncbi.nlm.nih.gov/genome/guide), POLK, and consequently COL4A3BP, maps to position 80300 Kbp. This, in addition to the other data presented above and in WO 00/50607, suggests that the expression products of the POLK and GPBP genes play a role in human autoimmunity.

We show here that each orientation of a 140 base pair fragment of the bi-directional promoter for POLK/COL4A3BP is highly homologous to DNA regions at the gene junctions of a variety of bi-directional promoters. The sequence homology found among different intergene regions transcribing structurally unrelated genes, as well as the TNF-induced coordinated expression of these genes, likely reflect a strategy to link the expression of proteins that are partners in complex biological programs. Furthermore, we have shown that this 140 base pair fragment and homologous regions in other bi-directional units contain the structural requirements to initiate transcription and to respond to TNF.

Anti-TNF based therapeutic approaches have been shown to be effective in several autoimmune conditions including rheumatoid arthritis and Crohn's disease and is presently at the stage of critical clinical trials [12,43]. Anti-TNF based therapy has been shown also to have important therapeutic effects on experimental allergic encephalomyelitis (EAE), an animal model for multiple sclerosis, however similar therapeutic approach in human clinical trials resulted in clinical worsening [12]. In our case, although the animals treated maintained the autoantibody levels one developed a GN more aggressive than untreated animals and mice in which anti-TNF treatment was extended for one additional month showed more abundant histological damage and very high GPBP/GPBPΔ26 ratios (not shown).

All the evidences above suggest that, in our model, the anti-TNF treatment is likely operating over the autoimmune response, and is very effective at inhibiting autoantibody production. However, likely because the cytokine is expected to be high in the pathogenic cascade and is known to be involved in various biological functions [12], anti-TNF treatment appears to have limitations. The coordinated expression of the multiple bi-directional promoters in response to TNF and the coordinated elevation of the CPBP/GPBPΔ26 and pol κ76/pol κ ratios in human cutaneous lupus suggest that bi-directional promoters are partners in apoptotic programs which become upregulated during autoimmune pathogenesis. Consequently, an intervention at the transcriptional level over common transacting factor(s) likely represent a way to achieve therapeutic effects on the autoimmune response with less site effects than anti-TNF based therapy.

### References

1. Saus, J. (1998) in Goodpasture's Syndrome: Encyclopedia of Immunology 2nd edn. Vol. 2, eds. Delves, P. J., & Roitt, I. M., (Academic Press Ltd., London), pp. 1005-1011.
2. Raya, A., Revert, F., Navarro, S., and Saus, J. (1999) Characterization of a novel type of serine/threonine kinase that specifically phosphorylates the human Goodpasture antigen J. Biol. Chem. 274, 12642-12649.
3. Raya, A., Revert-Ros, F., Martinez-Martinez, P., Navarro, S., Roselló, E., Vieites, B., Granero, F., Forteza, J. and Saus, J. (2000) Goodpasture antigen-binding protein, the kinase that phosphorylates the Goodpasture antigen, is an alternatively spliced variant implicated in autoimmune pathogenesis. J. Biol. Chem. 275, 40392-40399.
4. Wagner, J., Gruz, P., Kim, S.-R., Yamada, M., Matsui, K., Fuchs, R. P. P. and Nohmi, T.(1999) The dinB gene encodes a novel E. coli DNA polymerase, DNA Pol IV, involved in mutagenesis. Mol. Cell 4, 281-286.
5. Gerlach, V. L., Aravind, L., Gotway, G., Schultz, R. A., Koonin, E. V. and Friedberg, E. C. (1999) Human and mouse homologs of E. coli DinB (DNA polymerase IV), members of the UmuC/DinB superfamily. Proc. Natl. Acad. Sci. USA 96, 11922-11927.
6. Johnson, R. E., Prakash, S.and Prakash, L.(2000) The human DINB1 gene encodes the DNA polymerase polθ. Proc. Natl. Acad. Sci. USA 97, 3838-3843.
7. Tang, M., Pham, P., Shen, X., Taylor, J.-S., O'Donnell, M., Woodgate, R. and Goodman, M. F. (2000) Roles of the E.coli DNA polymerases IV and V in lesion-targeted and untargeted SOS mutagenesis. Nature 404, 1014-1018.
8. Ohashi, E., Bebenek, K., Matsuda, T., Feaver, W. J., Gerlach, V. L., Friedberg, E. C., Ohmori, H. and Kunkel, T. A.(2000) Fidelity and processivity of DNA synthesis by DNA polymerase κ, the product of the human DINB1 gene. J. Biol. Chem. 275, 39678-39684.
9.Zhang, Y., Yuan, F., Xin, H., Wu, X., Rajpal, D. K., Yang, D. and Wang, Z.(2000) Human DNA polymerase κ synthesizes DNA with extraordinarily low fidelity. Nucleic Acids Res. 28, 4147-4156.
10. Zhang, Y., Yuan, F., Wu, X., Wang, M., Rechkoblit, O., Taylor, J.-S., Geacintov, N. E. and Wang, Z.(2000) Error-free and error-prone lesion bypass by human DNA polymerase κ in vitro. Nucleic Acids Res. 28, 4138-4146.
11. Gerlach, V. L., Feaver, W. J., Fischhaber, P. L., and Friedberg, E. C.(2001) Purification and characterization of pol κ, a DNA polymerase encoded by the human DINB1 gene. J. Biol. Chem. 276, 92-98.
12. Oppenheim, J. J and Feldmann, M.(2001) in Cytokine Reference. A compedium of cytokines and other mediators of host defense Vol. 1 (Academic Press Ltd.,London), pp 413-447.
13. Nadal, M., Moreno, S., Pritchard, M., Preciado, M. A., Estivill, X., and Ramos-Arroyo, M. A.(1997) Down syndrome: characterisation of a case with partial trisomy of chromosome 21 owing to a paternal balanced translocation (15;21) (q26;q22.1) by FISH. J. Med. Genet. 34, 50-54.
14. Needleman, S. B. and Wunsch, C. D. (1970) A general method applicable to the search for similarities in the amino acid sequence of two proteins. J. Mol. Biol. 48, 443-453.
15. López-Hoyos, M., Diez, M. A., Buelta, L., Izui, S., Merino J., and Merino, R.(1999) Overexpression of human Bcl-2 in germinal center B cells induce a new and severe autoimmune syndrome in (C57BL/6×NZW)F1 mice. Arthritis Rheum. 42(9):S393.
16. Gonzalez M, Schurmans S, Ramos A, Merino R, Lambert P-H and Merino J. (1995) CD4+ T cells determine the ability of spleen cells from F1 hybrid mice to induce neonatal tolerance to alloantigens and autoimmunity in parental mice. Eur. J. Immunol. 25: 1760-1764.
17. López-Hoyos, M., Carrió, R., Merino, R., Buelta, L., Izui, S., Núñdez, G., and Merino, J.(1996). Constitutive expression of Bcl-2 in B cells causes a lethal form of lupuslike autoimmune disease after induction of neonatal tolerance to H-2^{b} alloantigens. J. Exp. Med. 183, 2523-2531.
18. Echtenacher B, Falk W, Mannel D A and Krammer P H (1990) Requirement of endogenous Tumor Necrosis Factor/Cachectin for recovery from experimental peritonitis. J. Immunol. 145, 3762-3766
19. Remick D, Manohar P, Bolgos G, Rodriguez J, Moldawer L, and Wollenberg G. (1995) Blockade of tumor necrosis factor reduces lipopolysaccharide lethality, but not the lethality of cecal ligation and puncture. Shock, 4, 89-95.
20. Wasylyk, B., Wasylyk, C., Augereau, P. and Chambon, P.(1983) The SV40 72 bp repeat preferentially potentiates transcription starting from proximal natural or substitute promoter elements. Cell 32, 503-514.
21. Hansen, U. and Sharp, P. (1983) Sequences controlling in vitro transcription of SV40 promoters. EMBO J. 2, 2293-2303.
22. Lavia, P., Macleod, D. and Bird, A. (1987) Coincident start sites for divergent transcripts at a randomly selected CpG-rich island of mouse. EMBO J. 6, 2773-2779.
23. Pöschl, E., Pollner, R. and Künh, K. (1988) The genes for the α1(IV) and a2(IV) chains of human basement membrane collagen type IV are arranged head-to-head and separated by a bi-directional promoter of unique structure. EMBO J. 7, 2687-2695.
24. Momota, R., Sugimoto, M., Oohashi, T., Kigasawa, K., Yoshioka, H. and Ninomiya, Y.(1998) Two genes, COL4A3 and COL4A4 coding for the human α3(IV) and α4(IV) collagen chains are arranged head-to-head on chromosome 2q36. FEBS Lett 424, 11-16.
25. Sugimoto, M., Oohashi, T., and Ninomiya, Y. (1994) The genes COL4A5 and COL4A6, coding for basement membrane collagen chains α5(IV) and α6(IV), are located head-to-head in close proximity on chromosome Xq22 and COL4A6 is transcribed from two alternative promoters. Proc. Natl. Acad. Sci. USA 91, 11679-11683.
26. Quinones, S., Bernal, D., Garcia-Sogo, M., Elena, S. F. and Saus, J. (1992) Exon/intron structure of the human α3(IV) gene encompassing the Goodpasture antigen (□3(IV)NC1). J. Biol. Chem. 267, 19780-19784.
27. Mariyama, M., Kalluri, R. , Hudson, B. G. and Reeders, S. T. (1992) The α4(V) chain of basement membrane collagen. J. Biol. Chem. 267, 1253-1258.
28. Sugimoto, M., Oohashi, T., Yoshioka, H., Matsuo, N., and Ninomiya, Y. (1993). cDNA isolation and partial gene structure of the human α4(IV)collagen chain. FEBS Lett 330, 122-128.
29. Oohashi, T., Ueki, Y., Sugimoto, M. and Ninomiya, Y.(1995). Isolation and structure of the COL4A6 gene encoding the human α6(IV) collagen chain and comparison with other type IV collagen genes. J. Biol. Chem. 270, 26863-26867.
30. Shimada, T, Fujii, H. and Lin, H. (1989) A 165-base pair sequence between the dihydrofolate reductase gene and the divergently transcribed upstream gene is sufficient for bi-directional transcriptional activity. J. Biol. Chem. 264, 20171-20174.
31. Shinya, E. and Shimada, T. (1994) Identification of two initiator elements in the bi-directional promoter of the human dihydrofolate reductase and mismatch repair protein 1 genes. Nucleic Acids Res. 22, 2143-2149.
32. O'Hanlon, T. P., Raben, N., and Miller F. W. (1995) A novel gene oriented in a head-to-head configuration with the human histidyl-tRNA synthetase (HRS) gene encodes an mRNA that predicts a polypeptide homologous to HRS. Biochem. Biophys. Res. Commun. 210, 556-566.
33. Tsui, H. W., Mok, S., Souza, L., Marttin, A., and Tsui, F. W. L.(1993) Transcriptional analyses of the gene region that encodes the human histidyl-tRNA synthetase: Identification of a novel bi-directional regulatory element. Gene 131, 201-208.
34. Gavalas, A. and Zalkin, H. (1995) Analysis of the chicken GPAT/AIRC bi-directional promoter for de novo purine nucleotide synthesis. J. Biol. Chem. 270, 2403-2410.
35. Wright, K. L., White, L. C., Kelly, A., Beck, S., Trowsdale, J., and Ting, J.P.-Y. (1995) Coordinate regulation of the human TAP1 and LMP2 genes from a shared bi-directional promoter. J. Exp. Med. 181, 1459-1471.
36. Ryan, M. T., Herd, S. M., Sbema, G., Samuel, M. M., Hoogenraad, N. J. and Hoj, P. B. (1997) The genes encoding mammalian chaperonin 60 and chaperonin 10 are linked head-to-head and share a bi-directional promoter. Gene 196, 9-17.
37. Brenner, V., Nyakatura, G., Rosenthal, A. and Platzer, M. (1997) Genomic organization of two novel genes on human Xq28:Compact head to head arrangement of IDHγ and TRAPδ is conserved in rat and mouse. Genomics 44, 8-14.
38. Casciola-Rosen, L. A., Anhalt, G. and Rosen, A. (1994) Autoantigens targeted in systemic lupus erythematosus are clustered in two populations of surface structures on apoptotic keratinocytes. J. Exp. Med. 179, 1317-1330.
39. Casciola-Rosen, L., & Rosen, A. (1997) Ultraviolet light-induced keratinocyte apoptosis: a potential mechanism for the induction of skin lesions and autoantibody production in LE.Lupus 6, 175-180.
40. Utz, P. J., and Anderson, P. (1998) Posttranslational protein modifications, apoptosis, and the bypass of tolerance to autoantigens. Arthritis & Rheum. 41, 1152-1160.
41. Pablos, J. L:, Santiago, B., Galindo, M., Carreira, P. E., Ballestin, C. and Gomez-Reino, J. J. (1999) Keratinocyte apoptosis and p53 expression in cutaneous lupus and dermatomyositis. J. Pathol. 188, 63-68.
42. The Multiple Sclerosis Genetics Group (1996) A complete genomic screen for multiple sclerosis underscores a role for the major histocompatability complex. Nature Genet.13, 469-471.
43. Felmann, M., Bondeson, J., Brennan, F. M., Foxwell, B. M., and Maini, R N.(1999).

The rationale for the current boom in anti-TNF□treatment. Is there an effective means to define therapeutic targets for drugs that provide all the benefits of anti-TNFα and minimise hazards? Ann. Rheum. Dis. 58 Suppl1, I27-31.

I claim:

1. An isolated nucleic acid consisting of a sequence that encodes a polypeptide with the amino acid sequence of SEQ ID NO:31.

2. The isolated nucleic acid of claim 1 wherein the nucleic acid consists of the sequence in SEQ ID NO:30.

3. A recombinant expression vector comprising the isolated nucleic acid sequence of claim 1.

4. A host cell transfected with the recombinant expression vector of claim 3.

5. A isolated polypeptide, consisting of the amino acid sequence of SEQ ID NO:31.

6. A method for detecting a polypeptide consisting of the amino acid sequence of SEQ ID NO:31, comprising
a) providing a protein sample to be screened;
b) contacting the protein sample to be screened with an antibody that selectively recognizes the polypeptide consisting of the amino acid sequence of SEQ ID NO:31 under conditions that promote antibody-polypeptide complex formation; and
c) detecting the formation of an antibody-polypeptide complexes, wherein the presence of the antibody-polypeptide complexes indicates the presence of the polypeptide consisting of the amino acid sequence of SEQ ID NO:31.

7. The method of claim 6, wherein detecting comprises a method selected from the group consisting of immunolocalization, immunofluorescence analysis, Western blot analysis, ELISAs, and nucleic acid expression library screening.

8. A method for detecting in a sample a nucleic sequence consisting of the sequence of SEQ ID NO:30, comprising
a) contacting the sample with one or more oligonucleotides that can be used to generate polymerase chain reaction products that selectively identify the nucleic acid sequence of SEQ ID NO:30;
b) carrying out a polymerase chain reaction to generate the polymerase chain reaction products; and
c) identifying the polymerase chain reaction products as being specific for the nucleic acid sequence of SEQ ID NO:30

9. The method of claim 8, wherein the detecting is carried out by a method selected from the group consisting of reverse transcription-polymerase chain reaction, and polymerase chain reaction.

10. A method for detecting an autoimmune condition in a patient, comprising
a) providing a tissue or body fluid sample from the patient;
b) providing a control tissue or body fluid sample in which no autoimmune condition is present; and
c) detecting an increase in pol κ76 RNA expression in the tissue or body fluid sample compared to the control sample, wherein the increase indicates the presence of an autoimmune condition.

11. A method for treating a patient with an autoimmune disorder or cancer, comprising modifying the expression or activity of pol κ76 in the patient.

12. The method of claim 11 wherein modifying the expression or activity of pol κ76 in the patient comprises administering a compound effective for inhibiting tumor necrosis factor induction of POLK gene expression.


SEQUENCE LISTING (102 sequences)

SEQ ID NO: 1 - DNA, 2389 bp, Homo sapiens
gcaggaagatggcggcggtagcggaggtgtgagtggacgcgggactcagcggccggattttctcttcccttcttttcccttttccttccctatttgaaattggcatcgagggggctaagttcgggtggcagcgccgggcgcaacgcaggggtcacggcgacggcggcggcggctgacggctggaagggtaggcttcattcaccgctcgtcctccttcctcgctccgctcggtgtcaggcgcggcggcggcgcggcgggcggacttcgtccctcctcctgctcccccccacaccggagcgggcactcttcgcttcgccatcccccgacccttcaccccgaggactgggcgcctcctccggcgcagctgagggagcgggggccggtctcctgctcggttgtcgagcctccatgtcggataatcagagctggaactcgtcgggctcggaggaggatccagagacggagtctgggccgcctgtggagcgctgcggggtcctcagtaagtggacaaactacattcatgggtggcaggatcgttgggtagttttgaaaaataatgctctgagttactacaaatctgaagatgaaacagagtatggctgcagaggatccatctgtcttagcaaggctgtcatcacacctcacgattttgatgaatgtcgatttgatattagtgtaaatgatagtgtttggtatcttcgtgctcaggatccagatcatagacagcaatggatagatgccattgaacagcacaagactgaatctggatatggatctgaatccagcttgcgtcgacatggctcaatggtgtccctggtgtctggagcaagtggctactctgcaacatccacctcttcattcaagaaaggccacagtttacgtgagaagttggctgaaatggaaacatttagagacatcttatgtagacaagttgacacgctacagaagtactttgatgcctgtgctgatgctgtctctaaggatgaacttcaaagggataaagtggtagaagatgatgaagatgactttcctacaacgcgttctgatggtgacttcttgcatagtaccaacggcaataaagaaaagttatttccacatgtgacaccaaaaggaattaatggtatagactttaaaggggaagcgataacttttaaagcaactactgctggaatccttgcaacactttctcattgtattgaactaatggttaaacgtgaggacagctggcagaagagactggataaggaaactgagaagaaaagaagaacagaggaagcatataaaaatgcaatgacagaacttaagaaaaaatcccactttggaggaccagattatgaagaaggccctaacagtctgattaatgaagaagagttctttgatgctgttgaagctgctcttgacagacaagataaaatagaagaacagtcacagagtgaaaaggtgagattacattggcctacatccttgccctctggagatgccttttcttctgtggggacacatagatttgtccaaaagccctatagtcgctcttcctccatgtcttccattgatctagtcagtgcctctgatgatgttcacagattcagctcccaggttgaagagatggtgcagaaccacatgacttactcattacaggatgtaggcggagatgccaattggcagttggttgtagaagaaggagaaatgaaggtatacagaagagaagtagaagaaaatgggattgttctggatcctttaaaagctacccatgcagttaaaggcgtcacaggacatgaagtctgcaattatttctggaatgttgacgttcgcaatgactgggaaacaactatagaaaactttcatgtggtggaaacattagctgataatgcaatcatcatttatcaaacacacaagagggtgtggcctgcttctcagcgagacgtattatatctttctgtcattcgaaagataccagccttgactgaaaatgaccctgaaacttggatagtttgtaatttttctgtggatcatgacagtgctcctctaaacaaccgatgtgtccgtgccaaaataaatgttgctatgatttgtcaaaccttggtaagcccaccagagggaaaccaggaaattagcagggacaacattctatgcaagattacatatgtagctaatgtgaaccctggaggatgggcaccagcctcagtgttaagggcagtggcaaagcgagagtatcctaaatttctaaaacgttttacttcttacgtccaagaaaaaactgcaggaaagcctattttgttctagtattaacaggtactagaagatatgttttatctttttttaactttatttgactaatatgactgtcaatactaaaatttagttgttgaaagtatttactatgtttttt

SEQ ID NO: 2 - DNA, 1304 bp, Homo sapiens
gacgaaccctccgggcttgcgggcccagacgtgagagagctttccgctgaagatgacgggcctgctttccagggcggcttgtcgaaagcccgggagcatctggccgcttccgcctcaaccatgggctggggttttgtgagctactagtgccaagggttttctttccaccagaccaccgctgtaaatctcgagggtcttactcattagaagttagaattcacatttgacgtttaaaggaagaatttccttagtaccttctcacaagcacgcacttcgcatttttagatttctagagtttgctttgtagaaagtaattttgaggttgtcagagaataaatgacgttagaaaggtttttaaagtaaaacaagaatgtgagatgatagcctgggattttctcttggttgtaaatgaatatcttactgagaaccacgttaaccatgcctgcccctcaaagataggaaaggttggatatatagaaactttctcgtattagaaataccgaagtgcagtggttttgtgtgtacaagggattaggcaataggaggctatttttgttttaagactagggttgaattagcagaaagaccaatagaagatctaacaactcttgtcagttgtcaaggataactttgattatgagactttgactttgtagcttcagtaatttcctctcgttagctattttaatatagtcgatttccttgtaattgccaagagtaaaatttgttattaaaccttagaaagagtactttcttactacaaggatgggacgataggagcgaaatttcgagtctaagggaaaacgctggccgagtgtggtggctcacgcctgtgatcccggcacttcgggaggccgaggtgggtggatcacctgaggccgggagtttgagaccagcctgggcggcagggtgggaccccgtctctactaaaaatacaaagattagccgagcatggtggtaggtgcctgtaactccagctctttatatcctggtttcaaatctaggcttgatgaccttctcccatatcccagtatcatatttttttcttcctgcatgggggattaattacgattctgaatggttggtagcatgaagctaggttatccctatcgtggcaatggatatttaagtaggcattgccaatatttatcttgctttcttttactttcttctttttctgaccatccacactccatttatattgatgagttcttttactaatatcaattattattatattatgctcatactgccatgtcttattctgcagctttgatccttaaggtgactttgcatatctgtct

SEQ ID NO: 3 - DNA, 955 bp, Homo sapiens
ggcatggttaacgtggttctcagtaagatattcatttacaaccaagagaaaatcccaggctatcatctcacattcttgttttactttaaaaacctttctaacgtcatttattctctgacaacctcaaaattactttctacaaagcaaactctagaaatctaaaaatgcgaagtgcgtgcttgtgagaaggtactaaggaaattcttcctttaaacgtcaaatgtgaattctaacttctaatgagtaagaccctcgagatttacagcggtggtctggtggaaagaaaacccttggcactagtagctcacaaaaccccagcccatggttgaggcggaagcggccagatgctcccgggctttcgacaagccgccctggaaagcaggcccgtcatcttcagcggaaagctctctcacgtctgggcccgcaagcccggagggttcgtcataaacacacaaggcaaggatagaagcgaggccgaggggctggtcacgcaactgtcaaacgaagcccacccaccgactgacaaggccccaaggggacaagcgatccccgcgcgggatactcacccgttacctcaggatcgcgactacaactcccaggaggctgcgcgagcgacggaccaacgcccttcccagaatgcagcacagctgcatccctaccccgccctctcctttctccgctcctcctgcttttctacccgtcgtcacccgggagagccggaggtagggttcgggaggaggatcccgaaggctcggcgtgtcgcgtcagacgccgggagggggacggggcggggagtagtgggggagaatgggaggacgaaggggaggggaaaggacaggggaggggagggtaaatagtgggccaggcaggaagatggcggcggtagcggaggtgtgagtggacgcgggactcagcggccggattttctcttcccttcttttcccttttccttccc

SEQ ID NO: 4 - DNA, 771 bp, Homo sapiens
tctagaaatctaaaaatgcgaagtgcgtgcttgtgagaaggtactaaggaaattcttcctttaaacgtcaaatgtgaattctaacttctaatgagtaagaccctcgagatttacagcggtggtctggtggaaagaaaacccttggcactagtagctcacaaaaccccagcccatggttgaggcggaagcggccagatgctcccgggctttcgacaagccgccctggaaagcaggcccgtcatcttcagcggaaagctctctcacgtctgggcccgcaagcccggagggttcgtcataaacacacaaggcaaggatagaagcgaggccgaggggctggtcacgcaactgtcaaacgaagcccacccaccgactgacaaggccccaaggggacaagcgatccccgcgcgggatactcacccgttacctcaggatcgcgactacaactcccaggaggctgcgcgagcgacggaccaacgcccttcccagaatgcagcacagctgcatccctaccccgccctctcctttctccgctcctcctgcttttctacccgtcgtcacccgggagagccggaggtagggttcgggaggaggatcccgaaggctcggcgtgtcgcgtcagacgccgggagggggacggggcggggagtagtgggggagaatgggaggacgaaggggaggggaaaggacaggggaggggagggtaaatagtgggccaggcaggaagatggcggcggtagcggaggtgtgagtggacgcgggactcagcggccg

SEQ ID NO: 5 - DNA, 771 bp, Homo sapiens
cggccgctgagtcccgcgtccactcacacctccgctaccgccgccatcttcctgcctggcccactatttaccctcccctcccctgtcctttcccctccccttcgtcctcccattctcccccactactccccgccccgtccccctcccggcgtctgacgcgacacgccgagccttcgggatcctcctcccgaaccctacctccggctctcccgggtgacgacgggtagaaaagcaggaggagcggagaaaggagagggcggggtagggatgcagctgtgctgcattctgggaagggcgttggtccgtcgctcgcgcagcctcctgggagttgtagtcgcgatcctgaggtaacgggtgagtatcccgcgcggggatcgcttgtccccttggggccttgtcagtcggtgggtgggcttcgtttgacagttgcgtgaccagcccctcggcctcgcttctatccttgccttgtgtgtttatgacgaaccctccgggcttgcgggcccagacgtgagagagctttccgctgaagatgacgggcctgctttccagggcggcttgtcgaaagcccgggagcatctggccgcttccgcctcaaccatgggctggggttttgtgagctactagtgccaagggttttctttccaccagaccaccgctgtaaatctcgagggtcttactcattagaagttagaattcacatttgacgtttaaaggaagaatttccttagtaccttctcacaagcacgcacttcgcatttttagatttctaga

SEQ ID NO: 6 - DNA, 140 bp, Homo sapiens
gggttcgggaggaggatcccgaaggctcggcgtgtcgcgtcagacgccgggagggggacggggcggggagtagtgggggagaatgggaggacgaaggggaggggaaaggacaggggaggggagggtaaatagtgggccag

SEQ ID NO: 7 - DNA, 140 bp, Homo sapiens
ctggcccactatttaccctcccctcccctgtcctttcccctccccttcgtcctcccattctcccccactactccccgccccgtccccctcccggcgtctgacgcgacacgccgagccttcgggatcctcctcccgaaccc

SEQ ID NO: 8 - DNA, 137 bp, Homo sapiens
gggctagtggcgaggctgagggcttcacgcaggtcccgacaggcagcgagcggaagggagcaagcggggatgccccggaacaggtggaatgcgcggggctgggggaagaggcgaggagggggcttgtccagtgccta

SEQ ID NO: 9 - DNA, 137 bp, Homo sapiens
taggcactggacaagccccctcctcgcctcttcccccagccccgcgcattccacctgttccggggcatccccgcttgctcccttccgctcgctgcctgtcgggacctgcgtgaagccctcagcctcgccactagccc

SEQ ID NO: 10 - DNA, 142 bp, Homo sapiens
tggccactccctccaccctgcgcagccacctccccaccgcgcagccacctccccaccgcacacccccaaacgccccacctccgaccgcaccccacttccccgcctgggcccccggaccttgggagcatcacctccttaaccc

SEQ ID NO: 11 - DNA, 142 bp, Homo sapiens
gggttaaggaggtgatgctcccaaggtccgggggcccaggcggggaagtggggtgcggtcggaggtggggcgtttgggggtgtgcggtggggaggtggctgcgcggtggggaggtggctgcgcagggtggagggagtggcca

SEQ ID NO: 12 - DNA, 371 bp, Homo sapiens
aagcggggcctcccgcagacgccggcgcgcctcccgttaatctgggcagggccgctggccactccctccaccctgcgcagccacctccccaccgcgcagccacctccccaccgcacacccccaaacgccccacctccgaccgcaccccacttccccgcctgggcccccggaccttgggagcatcacctccttaaccccttaccctggatccgcgcccacctgcccctcaggcgcccagccctttctcgcctcctgggcacgatgcccgggtagaagggacactgcctggtaagttgggagggagggggtatgagggcgggacctgagccacgtcttccctcccttgaagccacaaccaaaaagcctgggtg

SEQ ID NO: 13 - DNA, 371 bp, Homo sapiens
cacccaggctttttggttgtggcttcaagggagggaagacgtggctcaggtcccgccctcataccccctccctcccaacttaccaggcagtgtcccttctacccgggcatcgtgcccaggaggcgagaaagggctgggcgcctgaggggcaggtgggcgcggatccagggtaaggggttaaggaggtgatgctcccaaggtccgggggcccaggcggggaagtggggtgcggtcggaggtggggcgtttgggggtgtgcggtggggaggtggctgcgcggtggggaggtggctgcgcagggtggagggagtggccagcggccctgcccagattaacgggaggcgcgccggcgtctgcgggaggccccgctt

SEQ ID NO: 14 - DNA, 140 bp, Homo sapiens
ctggtgcccaattttctccatcacgcacacccttctcgcctctccctgcctcctgcctttccacttgcaccagttttcccaccccagcctcagggcggggctgcctcgtcacttgtctcggggcagatctgccctacaca

SEQ ID NO: 15 - DNA, 140 bp, Homo sapiens
tgtgtagggcagatctgccccgagacaagtgacgaggcagccccgccctgaggctggggtgggaaaactggtgcaagtggaaaggcaggaggcagggagaggcgagaagggtgtgcgtgatggagaaaattgggcaccag

SEQ ID NO: 16 - DNA, 140 bp, Homo sapiens
gggctgggggggcggggcttgtgggtaaggcgggcggaggcggggaccctccgcccgatgatagggctggaggaggaagcggcgggctgaagaaggggaaggtgggaagagcccagccggggctacaaattgggtgaagc

SEQ ID NO: 17 - DNA, 140 bp, Homo sapiens
gcttcacccaatttgtagccccggctgggctcttcccaccttccccttcttcagcccgccgcttcctcctccagccctatcatcgggcggagggtccccgcctccgcccgccttacccacaagccccgcccccccagccc

SEQ ID NO: 18 - DNA, 140 bp, Homo sapiens
ggttgccggtgcagtctaaaactgtggcggagtgatactcaaattcccttgtgctggtgaggaggggggccttgcacggggaagagagggaggaaagtagatctgtaggaattgagtgaagaaaaagtttgcaagtctgg

SEQ ID NO: 19 - DNA, 140 bp, Homo sapiens
ccagacttgcaaactttttcttcactcaattcctacagatctactttcctccctctcttccccgtgcaaggcccccctcctcaccagcacaagggaatttgagtatcactccgccacagttttagactgcaccggcaacc

SEQ ID NO: 20 - DNA, 140 bp, Homo sapiens
cggggctgtctgctgtcaatcatcccccctaccttgggcagccggtagtctttctcactttcaggcacctttccacacaacagccctaagtatctccacagcttcacacacagccccttagagacctatacgctaagacc

SEQ ID NO: 21 - DNA, 140 bp, Homo sapiens
ggtcttagcgtataggtctctaaggggctgtgtgtgaagctgtggagatacttagggctgttgtgtggaaaggtgcctgaaagtgagaaagactaccggctgcccaaggtaggggggatgattgacagcagacagccccg

SEQ ID NO: 22 - DNA, 140 bp, Homo sapiens
cggagccctggtgtcccggcgcactgcagccacactcccgggccgcgcgctcccgccgcctcttacccgcgccgcagggtcctcccctttgaggcgccgcccgcgcaccgccgggcgggagggggcagcgccaacaaatt

SEQ ID NO: 23 - DNA, 140 bp, Homo sapiens
aatttgttggcgctgccccctcccgcccggcggtgcgcgggcggcgcctcaaaggggaggaccctgcggcgcgggtaagaggcggcgggagcgcgcggcccgggagtgtggctgcagtgcgccgggacaccagggctccg

SEQ ID NO: 24 - DNA, 140 bp, Homo sapiens
gaggtgcgcaaacgcccgagttttccctggtgcgcgggttccgcctttgcagtgccctccacccttcctggtgtctgacccgcctccttcccaggccttttgttcctgtcccggaaagccggcgtcctgccgcgcgatgc

SEQ ID NO: 25 - DNA, 140 bp, Homo sapiens
gcatcgcgcggcaggacgccggctttccgggacaggaacaaaaggcctgggaaggaggcgggtcagacaccaggaagggtggagggcactgcaaaggcggaacccgcgcaccagggaaaactcgggcgtttgcgcacctc

SEQ ID NO: 26 - DNA, 140 bp, Homo sapiens
cgcggcaccgcgtgtgcaggcagctcccacccacttcccgtcagcccgggccctgcaatctgcacaccctgcgcgcgagccccgcccctccctacccgcgcagggtgtgctagcgcgctcagccctctccggccggctta

SEQ ID NO: 27 - DNA, 140 bp, Homo sapiens
taagccggccggagagggctgagcgcgctagcacaccctgcgcgggtagggaggggcggggctcgcgcgcagggtgtgcagattgcagggcccgggctgacgggaagtgggtgggagctgcctgcacacgcggtgccgcg

SEQ ID NO: 28 - DNA, 138 bp, Homo sapiens
cggaactccaggttgtcgccgcccgcaccctccagctggaccgcagaggaggaaggcccactcgggggtcgcaggagccggggggaggtggtgcgggaaggccgcgtacctgcggggcggcggcaaggcgtgcgctcg

SEQ ID NO: 29 - DNA, 138 bp, Homo sapiens
cgagcgcacgccttgccgccgccccgcaggtacgcggccttcccgcaccacctccccccggctcctgcgacccccgagtgggccttcctcctctgcggtccagctggagggtgcgggcggcgacaacctggagttccg

SEQ ID NO: 30 - DNA, 2026 bp, Homo sapiens
  CDS (1)..(2016)
atggatagcacaaaggagaagtgtgacagttacaaagatgatcttctgcttaggatgggacttaatgataataaagcaggaatggaaggattagataaagagaaaattaacaaaattataatggaagccacgaaggggtccagattttatggaaatgagctcaagaaagaaaagcaagtcaaccaacgaattgaaaatatgatgcaacaaaaagctcaaatcaccagccaacagctaagaaaagcacaattacaggttgacagatttgcaatggaattagaacaaagccgaaatttgagcaataccatagtgcacattgacatggatgctttctatgcagctgtagaaatgagggacaatccagaattgaaggataaacccattgctgtaggatcaatgagtatgctgtctacttcaaattaccatgcaaggagatttggtgttcgtgcagccatgccaggatttattgctaagaggctgtgcccacaacttataatagtgccccccaactttgacaaataccgagctgtgagtaaagaggttaaggaaatacttgctgattatgatcccaattttatggccatgagtcttgatgaagcctacttgaatataacaaagcacttagaagaaagacaaaattggcctgaggataaaagaaggtatttcatcaaaatgggaagctctgtagaaaatgataatccaggaaaggaagttaataaactgagtgagcatgaacgatccatctctccactactttttgaagagagtccttctgatgtgcagcccccaggagatcctttccaagtgaactttgaagaacaaaacaatcctcaaatactccaaaactcagttgtttttggaacatcagcccaggaagtggtaaaggaaattcgtttcagaattgagcagaaaacaacactgacagccagtgcaggtgttcggatatctagttttcccaatgaagaggacaggaaacaccaacaaaggagcattattggctttttacaggctggaaaccaagccctgtcagccactgagtgtacattagagaaaactgacaaagataagtttgtaaaacctctagaaatgtctcataagaagagtttctttgataaaaaacgatcagaaaggaaatggagtcaccaagatacatttaaatgtgaagccgtgaataaacaaagtttccagacatcacaaccattccaagttttaaagaagaagatgaatgagaatttggaaatatcagagaattcagatgactgtcagatacttacctgtcctgtttgctttagggctcaagggtgcatcagtctggaagccttgaataaacatgtagatgaatgtcttgatggaccttcaatcagtgaaaactttaaaatgttctcgtgttcacatgtttctgctaccaaagttaacaagaaagaaaatgttcctgcttcttcactttgtgagaagcaagattatgaagcccatccaaaaattaaagaaatatcttcagtagattgtatagctttagtagatactatagataactcatctaaagcagaaagcatagatgctttaagtaataagcatagcaaggaagaatgttctagtctcccaagcaagtcttttaatattgaacactgtcatcagaattcttcttctactgtttcattggaaaacgaagatgttggatcatttagacaagaataccgccagccttacttatgtgaagtgaaaacaggccaagctctagtttgtcctgtttgtaacgtagaacaaaagacttcagatctaaccctgttcaatgtgcatgtggatgtttgcttaaataaaagttttatccaagaattaagaaaggataaatttaacccagttaatcaacccaaagaaagctccagaagtactggtagctcaagtggagtacagaaggctgtaacaagaacaaaaaggccaggattgatgacaaagtactcaacatcaaagaaaataaaaccaaacaatcccaaacatacccttgatatattttttaagtaagtcgacc

SEQ ID NO: 31 - protein, 672 aa, Homo sapiens
MDSTKEKCDSYKDDLLLRMGLNDNKAGMEGLDKEKINKIIMEATKGSRFYGNELKKEKQVNQRIENMMQQKAQITSQQLRKAQLQVDRFAMELEQSRNLSNTIVHIDMDAFYAAVEMRDNPELKDKPIAVGSMSMLSTSNYHARRFGVRAAMPGFIAKRLCPQLIIVPPNFDKYRAVSKEVKEILADYDPNFMAMSLDEAYLNITKHLEERQNWPEDKRRYFIKMGSSVENDNPGKEVNKLSEHERSISPLLFEESPSDVQPPGDPFQVNFEEQNNPQILQNSVVFGTSAQEVVKEIRFRIEQKTTLTASAGVRISSFPNEEDRKHQQRSIIGFLQAGNQALSATECTLEKTDKDKFVKPLEMSHKKSFFDKKRSERKWSHQDTFKCEAVNKQSFQTSQPFQVLKKKMNENLEISENSDDCQILTCPVCFRAQGCISLEALNKHVDECLDGPSISENFKMFSCSHVSATKVNKKENVPASSLCEKQDYEAHPKIKEISSVDCIALVDTIDNSSKAESIDALSNKHSKEECSSLPSKSFNIEHCHQNSSSTVSLENEDVGSFRQEYRQPYLCEVKTGQALVCPVCNVEQKTSDLTLFNVHVDVCLNKSFIQELRKDKFNPVNQPKESSRSTGSSSGVQKAVTRTKRPGLMTKYSTSKKIKPNNPKHTLDIFFK

SEQ ID NO: 32 - DNA, 1335 bp, Homo sapiens
tgagagagctttccgctgaagatgacgggcctgctttccagggcggcttgtcgaaagcccgggagcatctggccgcttccgcctcaaccatgggctggggttttgtgagctactagtgccaagggttttctttccaccagaccaccgctgtaaatctcgagggtcttactcattagaagttagaattcacatttgacgtttaaaggaagaatttccttagtaccttctcacaagcacgcacttcgcatttttagatttctagagtttgctttgtagaaagtaattttgaggttgtcagagaataaatgacgttagaaaggtttttaaagtaaaacaagaatgtgagatgatagcctgggattttctcttggttgtaaatgaatatcttactgagaaccacgttaaccatgcctgcccctcaaagataggaaaggttggatatatagaaactttctcgtattagaaataccgaagtgcagtggttttgtgtgtacaagggattaggcaataggaggctatttttgttttaagactagggttgaattagcagaaagaccaatagaagatctaacaactcttgtcagttgtcaaggataactttgattatgagactttgactttgtagcttcagtaatttcctctcgttagctattttaatatagtcgatttccttgtaattgccaagagtaaaatttgttattaaaccttagaaagagtactttcttactacaaggatgggacgataggagcgaaatttcgagtctaagggaaaacgctggccgagtgtggtggctcacgcctgtaatcccagcacttcgggaggccgaggtgggtggatcacctgaggccgggagtttgagaccagcctgggcaacaagatttttcttcatccctttactttgagtctgtggatgtcattgcatgtgatatgggtctcctgaagacagcataccattggattttgcttctttatccaagttatcattctgtcttttaattggggtgtgcattcaagataagtttataccatggatagcacaaaggagaagtgtgacagttacaaagatgatcttctgcttaggatgggacttaatgataataaagcaggaatggaaggattagataaagagaaaattaacaaaattataatggaagccacgaaggggtccagattttatggaaatgagctcaagaaagaaaagcaagtcaaccaacgaattgaaaatatgatgcaacaaaaagctcaaatcaccagccaacagctaagaaaagcacaattacaggttgacagatttgcaatggaattagaacaaagccgaaatttgagcaatacca

SEQ ID NO: 33 - DNA, 105 bp, Mus musculus
gggagcgtcgcgagccgccgggaggggcccggggcggggtggagggaggatgggaggacggaggggagggagctgagagaggagggagggtaaatagtggacccg

SEQ ID NO: 34 - DNA, 105 bp, Mus musculus
cgggtccactatttaccctccctcctctctcagctccctcccctccgtcctcccatcctccctccaccccgccccgggcccctcccggcggctcgcgacgctccc

SEQ ID NO: 35 - DNA, 140 bp, Homo sapiens
ccctgcttatatagatgaccccctccccgagactctgacagacccaggtcacaggcagtcctcacctgctcctgacacccccggcccctcagtgctgctctctctagccaccgagctgaagtactgaggagcccctacct

SEQ ID NO: 36 - DNA, 140 bp, Homo sapiens
aggtaggggctcctcagtacttcagctcggtggctagagagagcagcactgaggggccgggggtgtcaggagcaggtgaggactgcctgtgacctgggtctgtcagagtctcggggagggggtcatctatataagcaggg

SEQ ID NO: 37 - DNA, 37 bp, Artificial Sequence
  Description of Artificial Sequence Primer ON-GPBP-6c
ctcgctcgcccagggaaggaaaagggaaaagaaggga

SEQ ID NO: 38 - DNA, 21 bp, Artificial Sequence
  Description of Artificial Sequence Primer ON-GPBP-14c
ctgcctggcccactatttacc

SEQ ID NO: 39 - DNA, 21 bp, Artificial Sequence
  Description of Artificial Sequence Primer ON-GPBP-18m
ggcatggttaacgtggttctc

SEQ ID NO: 40 - DNA, 30 bp, Artificial Sequence
  Description of Artificial Sequence Primer ON-XbaG/Bpro1m
gactctagagggttcgggaggaggatcccg

SEQ ID NO: 41 - DNA, 30 bp, Artificial Sequence
  Description of Artificial Sequence Primer ON-XbaG/Bpro1c
gactctagactggcccactatttaccctcc

SEQ ID NO: 42 - DNA, 30 bp, Artificial Sequence
  Description of Artificial Sequence Primer ON-SP1Del
cgccgggagggggacgtagtgggggagaat

SEQ ID NO: 43 - DNA, 30 bp, Artificial Sequence
  Description of Artificial Sequence Primer ON-TATADel
caggggaggggaggggtgggccagtctaga

SEQ ID NO: 44 - DNA, 22 bp, Artificial Sequence
  Description of Artificial Sequence Primer ON-DIN2c
ggattattgcacttgccttcac

SEQ ID NO: 45 - DNA, 27 bp, Artificial Sequence
  Description of Artificial Sequence Primer ON-DIN5′m
aaaggatccatggatagcacaaaggag

SEQ ID NO: 46 - DNA, 36 bp, Artificial Sequence
  Description of Artificial Sequence Primer ON-DIN-THc
aaaaaagtcgacttacttaaaaaatatatcaagggt

SEQ ID NO: 47 - DNA, 21 bp, Artificial Sequence
  Description of Artificial Sequence Primer ON-DINB1-R2
tggtattgctcaaatttcggc

SEQ ID NO: 48 - DNA, 18 bp, Artificial Sequence
  Description of Artificial Sequence Primer ON-GPBP-39c
tgagagagctttccgctg

SEQ ID NO: 49 - DNA, 29 bp, Artificial Sequence
  Description of Artificial Sequence Primer ON-LMPTAP1m
atgtctagatgtgtagggcagatctgccc

SEQ ID NO: 50 - DNA, 29 bp, Artificial Sequence
  Description of Artificial Sequence Primer ON-LMPTAP1c
atgtctagactggtgcccaattttctcca

SEQ ID NO: 51 - DNA, 29 bp, Artificial Sequence
  Description of Artificial Sequence Primer ON-HSP1m
atgtctagataagccggccggagagggct

SEQ ID NO: 52 - DNA, 29 bp, Artificial Sequence
  Description of Artificial Sequence Primer ON-HSP1c
atgtctagacgcggcaccgcgtgtgcagg

SEQ ID NO: 53 - DNA, 31 bp, Artificial Sequence
  Description of Artificial Sequence Primer ON-SA3A4m
gactctagagggttaaggaggtgatgctccc

SEQ ID NO: 54 - DNA, 32 bp, Artificial Sequence
  Description of Artificial Sequence Primer ON-SA3A4c
gactctagatggccactccctccaccctgcgc

SEQ ID NO: 55 - DNA, 32 bp, Artificial Sequence
  Description of Artificial Sequence Primer ON-INGA3A4m
gactctagacacccaggctttttggttgtggc

SEQ ID NO: 56 - DNA, 31 bp, Artificial Sequence
  Description of Artificial Sequence Primer ON-INGA3A4c
gactctagaaagcggggcctcccgcagacgc

SEQ ID NO: 57 - DNA, 29 bp, Artificial Sequence
  Description of Artificial Sequence Primer ON-S2A3A4m
atgtctagataggcactggacaagccccc

SEQ ID NO: 58 - DNA, 29 bp, Artificial Sequence
  Description of Artificial Sequence Primer ON-S2A3A4c
atgtctagagggctagtggcgaggctgag

SEQ ID NO: 59 - DNA, 20 bp, Artificial Sequence
  Description of Artificial Sequence Primer ON-IDH-F1
cacagagggcgagtacagca

SEQ ID NO: 60 - DNA, 21 bp, Artificial Sequence
  Description of Artificial Sequence Primer ON-IDH-R1
tgatcttcaggctctccacca

SEQ ID NO: 61 - DNA, 20 bp, Artificial Sequence
  Description of Artificial Sequence Primer ON-TRAPD-F1
gggtccagaacatggctctc

SEQ ID NO: 62 - DNA, 20 bp, Artificial Sequence
  Description of Artificial Sequence Primer ON-TRAPD-R1
acatcctggcctcgagtgac

SEQ ID NO: 63 - DNA, 21 bp, Artificial Sequence
  Description of Artificial Sequence Primer ON-LMP2-F2
gcagcatataagccaggcatg

SEQ ID NO: 64 - DNA, 20 bp, Artificial Sequence
  Description of Artificial Sequence Primer ON-LMP2-R2
tggccagagcaatagcgtct

SEQ ID NO: 65 - DNA, 19 bp, Artificial Sequence
  Description of Artificial Sequence Primer ON-TAP1-F2
gccgcctcactgactggat

SEQ ID NO: 66 - DNA, 21 bp, Artificial Sequence
  Description of Artificial Sequence Primer ON-TAP1-R2
tcgagtgaaggtatcggctga

SEQ ID NO: 67 - DNA, 19 bp, Artificial Sequence
  Description of Artificial Sequence Primer ON-DHFR-F1
cctgtggaggaggaggtgg

SEQ ID NO: 68 - DNA, 21 bp, Artificial Sequence
  Description of Artificial Sequence Primer ON-DHFR-R1
ccgattcttccagtctacggg

SEQ ID NO: 69 - DNA, 21 bp, Artificial Sequence
  Description of Artificial Sequence Primer ON-MSH3-F1
tgggtaaaggttggaagcaca

SEQ ID NO: 70 - DNA, 21 bp, Artificial Sequence
  Description of Artificial Sequence Primer ON-MSH3-R1
aaaaggagagtgaaagcggct

SEQ ID NO: 71 - DNA, 19 bp, Artificial Sequence
  Description of Artificial Sequence Primer ON-HO3-F2
gagctgttgtccctccgct

SEQ ID NO: 72 - DNA, 20 bp, Artificial Sequence
  Description of Artificial Sequence Primer ON-HO3-R2
ggccagataacgagcaaagg

SEQ ID NO: 73 - DNA, 21 bp, Artificial Sequence
  Description of Artificial Sequence Primer ON-HARS-F2
aggtggcgaaactcctgaaac

SEQ ID NO: 74 - DNA, 20 bp, Artificial Sequence
  Description of Artificial Sequence Primer ON-HARS-R2
tgctttcatcaggacccagc

SEQ ID NO: 75 - DNA, 21 bp, Artificial Sequence
  Description of Artificial Sequence Primer ON-Hsp10-F1
ggagggagtaatggcaggaca

SEQ ID NO: 76 - DNA, 21 bp, Artificial Sequence
  Description of Artificial Sequence Primer ON-Hsp10-R1
agcagcactcctttcaaccaa

SEQ ID NO: 77 - DNA, 21 bp, Artificial Sequence
  Description of Artificial Sequence Primer ON-Hsp60-F1
gcctttggtcataatcgctga

SEQ ID NO: 78 - DNA, 21 bp, Artificial Sequence
  Description of Artificial Sequence Primer ON-Hsp60-R1
tgccacaacctgaagaccaac

SEQ ID NO: 79 - DNA, 21 bp, Artificial Sequence
  Description of Artificial Sequence Primer ON-COL4A1-F1
gctctacgtgcaaggcaatga

SEQ ID NO: 80 - DNA, 20 bp, Artificial Sequence
  Description of Artificial Sequence Primer ON-COL4A1-R1
attgtgctgaacttgcgcag

SEQ ID NO: 81 - DNA, 20 bp, Artificial Sequence
  Description of Artificial Sequence Primer ON-COL4A2-F1
gaaaagggtgacgtagggca

SEQ ID NO: 82 - DNA, 21 bp, Artificial Sequence
  Description of Artificial Sequence Primer ON-COL4A2-R1
ggtgtctgatggaatcccgtt

SEQ ID NO: 83 - DNA, 21 bp, Artificial Sequence
  Description of Artificial Sequence Primer ON-GP-F1
ggagacagtggatcacctgca

SEQ ID NO: 84 - DNA, 21 bp, Artificial Sequence
  Description of Artificial Sequence Primer ON-GP-R1
tgctgtggtttgactgtgtcg

SEQ ID NO: 85 - DNA, 21 bp, Artificial Sequence
  Description of Artificial Sequence Primer ON-COL4A4-F1
cttgccttcccgtatttagca

SEQ ID NO: 86 - DNA, 21 bp, Artificial Sequence
  Description of Artificial Sequence Primer ON-COL4A4-R1
ggatctgtcgtttctctgggc

SEQ ID NO: 87 - DNA, 20 bp, Artificial Sequence
  Description of Artificial Sequence Primer ON-COL4A5-F1
catcgaatgtcatgggaggg

SEQ ID NO: 88 - DNA, 21 bp, Artificial Sequence
  Description of Artificial Sequence Primer ON-COL4A5-R1
agttgccagccaaaagctgta

SEQ ID NO: 89 - DNA, 21 bp, Artificial Sequence
  Description of Artificial Sequence Primer ON-COL4A6-F1
tttgggctagactaccggaca

SEQ ID NO: 90 - DNA, 20 bp, Artificial Sequence
  Description of Artificial Sequence Primer ON-COL4A6-R1
tctctatggacccgagggct

SEQ ID NO: 91 - DNA, 19 bp, Artificial Sequence
  Description of Artificial Sequence Primer ON-GPBP-F1
ctgaatccagcttgcgtcg

SEQ ID NO: 92 - DNA, 20 bp, Artificial Sequence
  Description of Artificial Sequence Primer ON-GPBP-R1
gcagagtagccacttgctcc

SEQ ID NO: 93 - DNA, 20 bp, Artificial Sequence
  Description of Artificial Sequence Primer ON-DinB1-F3
gccccccaactttgacaaat

SEQ ID NO: 94 - DNA, 21 bp, Artificial Sequence
  Description of Artificial Sequence Primer ON-DinB1-R3
gcttcatcaagactcatggcc

SEQ ID NO: 95 - DNA, 19 bp, Artificial Sequence
  Description of Artificial Sequence Primer ON-hGAPDH-F1
gaaggtgaaggtcggagtc

SEQ ID NO: 96 - DNA, 20 bp, Artificial Sequence
  Description of Artificial Sequence Primer ON-hGAPDH-R1
gaagatggtgatgggatttc

SEQ ID NO: 97 - DNA, 22 bp, Artificial Sequence
  Description of Artificial Sequence Primer ON-GPBP-26-1F
gctgttgaagctgctcttgaca

SEQ ID NO: 98 - DNA, 24 bp, Artificial Sequence
  Description of Artificial Sequence Primer ON-mGPBP-26-1R
ccatttcttcaaccttttgtacaa

SEQ ID NO: 99 - DNA, 21 bp, Artificial Sequence
  Description of Artificial Sequence Primer ON-GPBPe26-1R
cttgggagctgaatctgtgaa

SEQ ID NO: 100 - DNA, 20 bp, Artificial Sequence
  Description of Artificial Sequence Primer ON-huDINB-76-F1
ccagtgcaggtgttcggata

SEQ ID NO: 101 - DNA, 21 bp, Artificial Sequence
  Description of Artificial Sequence Primer ON-huDINB-76-R1
tttccagcctgtaaaaagcca

SEQ ID NO: 102 - DNA, 23 bp, Artificial Sequence
  Description of Artificial Sequence Primer ON-hGPBP-26-1R
ccatctcttcaaccttttggaca